(19) United States
(12) Patent Application Publication
(10) Pub. No.: US 2025/0034222 A1
(43) Pub. Date: Jan. 30, 2025

(54) IL-38 VARIANTS

(71) Applicants: Monash University, Clayton (AU); HUdson Institute of Medical Research, Clayton (AU)

(72) Inventors: Claudia Annelie Nold, Clayton (AU); Andrew Malcolm Ellisdon, Clayton (AU); Marcel Friedrich Nold, Clayton (AU); James Whisstock, Clayton (AU)

(73) Assignees: Monash University, Clayton (AU); Hudson Institute of Medical Research, Clayton (AU)

(21) Appl. No.: 18/717,170

(22) PCT Filed: Dec. 7, 2022
(86) PCT No.: PCT/AU2022/051464

§ 371 Date: Jun. 6, 2024

(30) Foreign Application Priority Data
Dec. 7, 2021 (AU) 2021903953

(51) Int. Cl. C07K 14/54; A61K 38/00; A61P 29/00; A61P 37/06; C12N 15/70

(52) U.S. Cl. CPC C07K 14/54; A61P 29/00; A61P 37/06; C12N 15/70; A61K 38/00; C07K 2319/30

(57) ABSTRACT

This invention relates to polypeptides, including variants of interleukin-38 (IL-38), and related therapeutics and compositions. The invention also relates to the use of the polypeptides and compositions in methods of treating inflammatory diseases or conditions. The present invention provides a monomeric polypeptide comprising an amino acid sequence of an IL-38 monomer, the amino acid sequence having a mutation or modification for preventing the peptide from forming a homodimer and favouring the formation of a stable monomer.

### RELATED APPLICATIONS

This application is a national stage filing under 35 U.S.C. § 371 of international application number PCT/AU2022/051464, filed Dec. 7, 2022, which claims the benefit of Australian application number AU 2021903953, filed Dec. 7, 2021, each of which is herein incorporated by reference in its entirety.

### REFERENCE TO AN ELECTRONIC SEQUENCE LISTING

The contents of the electronic sequence listing (M151170003US00-SEQ-KZM.xml; Size: 29,810 bytes; and Date of Creation: Jun. 4, 2024) are herein incorporated by reference in their entirety.

### FIELD OF THE INVENTION

This invention relates to polypeptides, including variants of interleukin-38 (IL-38), and related therapeutics and compositions. The invention also relates to the use of the polypeptides and compositions in methods of treating inflammatory diseases and conditions.

### BACKGROUND OF THE INVENTION

Inflammation plays a fundamental role in host defences and the progression of immune-mediated diseases. The inflammatory response is initiated in response to tissue injury (e.g., trauma, ischemia, and foreign particles) and infection by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events result in swelling, redness, warmth (altered heat patterns), and pus formation at the site of injury.

An interplay between the humoral and cellular immune elements in the inflammatory response enables the elimination of harmful agents and the initiation of the repair of damaged tissue. When this interplay is disrupted, the inflammatory response may result in considerable damage to normal tissue of uncontrolled inflammatory responses, clinical intervention is needed to prevent tissue damage and organ dysfunction.

Interleukin-38 (IL-38) is a member of the IL-1 family of cytokines, which is a heterogeneous group of proteins involved in the regulation of immunity and inflammation. IL-1 family cytokines exhibit a broad spectrum of functions in immunity, including the induction of Th1 and Th2 inflammation, as well as mediating anti-inflammatory effects. IL-38 (previously referred to as IL1F10) is the most recently-identified member of the IL-1 family. IL-38 has sequence homology with IL-1RA and has therefore been proposed to act as an IL-1 receptor antagonist. IL-38 has been shown to bind to IL-1R6 and thereby reduce cytokine production in response to C. albicans stimulation. Although the precise function of IL-38 remains to be elucidated, genome-wide association studies suggest an association of IL-38 polymorphisms with various inflammatory pathologies including spondyloarthritis, rheumatoid arthritis and psoriatic arthritis. Increased IL-38 expression has been reported in systemic lupus erythematosus (SLE), a severe multi-system autoimmune disease, with a strong association between IL-38 expression and SLE severity.

There exists a need for improved compositions and methods of treatment which can be used in modulating inflammatory pathways.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a polypeptide comprising an amino acid sequence of an IL-38 polypeptide, the amino acid sequence having a mutation or modification that reduces the capacity of the polypeptide to form a homodimer.

In another aspect, the present invention provides a polypeptide comprising an amino acid sequence of an IL-38 polypeptide, the amino acid sequence having a mutation or modification that increases the stability of the monomer and thereby reduces the likelihood that the polypeptide will form a homodimer.

Preferably, the mutation or modification reduces or prevents the polypeptide from forming a dimerization interface that enables dimerization of IL-38 monomers. Typically, the mutation or modification is located in a region of the polypeptide that has the same amino acid sequence as the amino acid sequence that forms the dimerization interface of an IL-38 monomer. Mutation or modification may be of an amino acid residue located in the domain-swap hinge region of IL-38, which is formed by the loop between the B4 and B5 strands, as herein defined. For example, mutation or modification may be of any residue which disrupts the alpha-helical structure that forms in this region of the IL-38 polypeptide, enabling formation of the IL-38 homodimer and which constitutes the domain-swap hinge region. Further, mutations or modification may be of any residue which stabilises the B4-85 turn or loop structure that forms in this region of the IL-38 monomer, preventing formation of a stable IL-38 homodimer and which constitutes the domain-swap hinge region in the homodimer.

In another aspect, the present invention provides a polypeptide comprising an amino acid sequence of an IL-38 polypeptide or fragment thereof, wherein the polypeptide has a reduced capacity to form a dimer compared to a polypeptide having the sequence of SEQ ID NO: 1. Preferably, the polypeptide exists in a slower equilibrium between monomer and dimer than a polypeptide having the sequence of SEQ ID NO: 1, preferably when tested under any conditions as described herein.

In another aspect, the present invention provides a polypeptide comprising an amino acid sequence of an IL-38 polypeptide or fragment thereof, wherein the polypeptide has the same capacity to form a dimer compared to a polypeptide having the sequence of SEQ ID NO: 1 or SEQ ID NO: 2.

In another aspect, the present invention provides a polypeptide comprising an amino acid sequence having at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with SEQ ID NO: 1, or with a functional variant thereof, wherein the polypeptide has a reduced capacity to form a dimer compared to a polypeptide having the sequence of SEQ ID NO: 1.

In another aspect, the present invention provides a polypeptide comprising an amino acid sequence having at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with SEQ ID NO: 1, or with a functional variant thereof, wherein the polypeptide has the same capacity to form a dimer compared to a polypeptide having the sequence of SEQ ID NO: 1 or SEQ ID NO: 2.

In another aspect, the present invention provides an IL-38 polypeptide that has a reduced capacity to form a dimer compared to a polypeptide having the sequence of SEQ ID NO: 1.

In certain embodiments, the IL-38 polypeptide of the invention is capable of forming a monomer that is as stable or more stable than the monomer encoded by SEQ ID NO: 2. In certain embodiments, the IL-38 polypeptide of the invention has a similar or decreased capacity to form a homodimer as compared to the protein encoded by SEQ ID NO: 2.

In another aspect, the present invention provides a polypeptide comprising an amino acid sequence of an IL-38 polypeptide or fragment thereof, wherein the polypeptide has a reduced capacity to form a dimer compared to a polypeptide having the sequence of SEQ ID NO: 1, wherein the polypeptide has a substitution or modification of at least one residue located in the dimerization interface of IL-38. Preferably, the mutation or modification is in a position equivalent to the loop between strands β4 and β5 of the IL-38 polypeptide. Preferably, the β4-β5 loop region consists of, or is equivalent to, residues 43 to 58 of SEQ ID NO: 1. More preferably, the substitution or modification is of a residue at a position, or at a position equivalent to, C43, 144, L45, P46, N47, R48, G49, L50, A51, R52, T53, K54, V55, P56, I57 or F58 of SEQ ID NO: 1 and, wherein the substitution or modification allows for stabilisation of the IL-38 monomeric form. For example, the substitution or modification is preferably one which promotes formation of an IL-38 monomer over an IL-38 homodimer (such that the equilibrium between formation of dimer and monomer is driven towards formation of the monomer). More preferably, the substitution or modification is one which allows for stabilisation of the IL-38 monomer, such that little or no homodimer is formed. Preferably the substitution is to an amino acid that prevents or reduces the capacity for formation of alpha-helical structure in the region between the β4 and β5 strands of the IL-38 monomer (for example, in the region corresponding to residues 43 to 58 of SEQ ID NO: 1). In other words, the substitution is one which reduces the likelihood of the formation of a domain-swap dimerization interface in the protein so that the formation of a homodimer is not favoured.

In another aspect, the present invention provides an IL-38 polypeptide that has a reduced capacity to form a dimer compared to a polypeptide having the sequence of SEQ ID NO: 1, wherein the polypeptide has a modification in the region corresponding to residues 43 to 58 of SEQ ID NO: 1. Modification includes, deleting the loop, shortening the loop, lengthening the loop, mutating one or more residues of the loop and/or chemically modifying one or more residues of the loop.

In another aspect, the present invention provides a polypeptide comprising an amino acid sequence of an IL-38 polypeptide or fragment thereof, wherein the polypeptide has a reduced capacity to form a dimer compared to a polypeptide having the sequence of SEQ ID NO: 1, wherein the polypeptide has a substitution or modification of a residue at, or at a position equivalent to C43, I44, L45, P46, N47, R48, G49, L50, A51, R52, T53, K54, V55, P56, 157 or F58 of SEQ ID NO: 1. Preferably, the mutation is any one of T53N, V55Q, or V55T.

In another aspect, the present invention provides a polypeptide comprising an amino acid sequence having at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with SEQ ID NO: 1, or with a functional variant thereof, wherein the amino acid residue at, or equivalent to:
position 43 in SEQ ID NO: 1 is not a Cysteine;
position 44 in SEQ IS NO: 1 is not an Isoleucine;
position 45 in SEQ ID NO: 1 is not a Leucine;
position 46 in SEQ ID NO: 1 is not a Proline;
position 47 in SEQ ID NO: 1 is not an Asparagine;
position 48 in SEQ ID NO: 1 is not an Arginine;
position 49 in SEQ ID NO: 1 is not a Glycine, preferably not a proline;
position 50 in SEQ ID NO: 1 is not a Leucine, preferably not an aspartate or glutamine;
position 51 in SEQ ID NO: 1 is not an Alanine;
position 52 in SEQ ID NO: 1 is not an Arginine;
position 53 in SEQ ID NO: 1 is not a Threonine, preferably not deleted;
position 54 in SEQ ID NO: 1 is not a Lysine;
position 55 in SEQ ID NO:1 is not a Valine;
position 56 in SEQ ID NO: 1 is not a Proline;
position 57 in SEQ ID NO: 1 is not an Isoleucine; and
position 58 in SEQ ID NO: 1 is not a Phenylalanine.

In certain embodiments, the amino acid residue is a non-conservative substitution relative to the amino acid that occurs in that position in SEQ ID NO: 1.

In any aspect, the mutation in the polypeptide is not a substitution at residue 51 from alanine to aspartic acid. Preferably, the mutation in the polypeptide is not a mutation at residue 51.

In any aspect, the polypeptide does not comprise or consist of the amino acid sequence of SEQ ID NO: 2. In any aspect, the polypeptide does not comprise or consist of the amino acid sequence of SEQ ID NO: 1. In any aspect, the polypeptide does not comprise or consist of the amino acid sequence of SEQ ID NO: 4 or 5.

In another aspect, the present invention provides a polypeptide that comprises a paralogous or an orthologous sequence to the sequence shown in SEQ ID NO: 1, wherein the polypeptide exists as a monomer or has a reduced capacity to form a dimer compared to a polypeptide having the sequence of SEQ ID NO: 1. Preferably, the polypeptide that comprises a paralogous or orthologous sequence to the sequence shown in SEQ ID NO: 1 has a substitution or modification is a residue that is located in a dimerization interface that is equivalent to the dimerization interface as described herein for SEQ ID NO: 1, preferably residues 43 to 58 of SEQ ID NO: 1.

In any embodiment, a polypeptide of the invention (including when provided in the form of a fusion protein as described elsewhere herein), is an anti-inflammatory polypeptide.

A polypeptide of the invention may be isolated, purified, substantially purified, enriched, synthetic or recombinant.

As used herein, the reduced capacity to form a dimer may refer to a reduced capacity to form a homodimer. Similarly, a reduced capacity to form a dimer may refer to an increased stability of the monomeric form of the protein compared to a protein having the sequence of SEQ ID NO: 1.

In another aspect, the invention also provides a polypeptide that comprises, consists essentially of or consists of an amino acid sequence of a IL-38 polypeptide or fragment thereof, wherein the amino acid sequence contains at least one mutation of, or modification to, a residue at, or at a position equivalent to, the dimer interface of SEQ ID NO: 1. The dimer interface in relation to a polypeptide having an amino acid sequence of SEQ ID NO: 1 includes the residues 43 to 58 of SEQ ID NO: 1. Typically, the polypeptide comprises an amino acid sequence that contains a mutation or modification of a residue at, or at a position equivalent to residues G49, L50, A51, T53 or V55 of SEQ ID NO: 1.

The present invention provides an isolated, recombinant or synthetic IL-38 polypeptide that does not have the capacity to form a dimer or has a reduced capacity to form a dimer. In this or any other aspect of the invention described herein, the capacity of a polypeptide to form a dimer may be determined by any method described herein, including size exclusion chromatography and multi-angle light scattering, analytical gel electrophoresis under non-denaturing conditions, analytical centrifugation, mass spectrometry or reverse phase high performance liquid chromatography (RP-HPLC).

Reduction in dimer formation can be compared to a reference polypeptide. The reference polypeptide will typically be a native, wild-type, unmodified or unmutated IL-38 polypeptide. Preferably, the reference polypeptide has an amino acid sequence of SEQ ID NO: 1. Alternatively, if the polypeptide of the invention has an amino acid sequence that is paralogous or orthologous to SEQ ID NO: 1, then the reference peptide is the native, wild-type, unmodified or unmutated paralogous or orthologous amino acid sequence.

In relation to any polypeptide of the invention described herein, the polypeptide may in addition have an N-terminal truncation. The truncation may be of at least residues equivalent to 1 to 20 of SEQ ID NO: 1, or part thereof. Preferably, the N-terminal truncation includes truncation of only residue 1, truncation of residues 1 to 2, or truncation of residues 1 to 3, or truncation of residues 1 to 6 of SEQ ID NO: 1. Preferably the truncation is of no more than up to the equivalent position of residues 1 to 6 of SEQ ID NO: 1. More preferably, the polypeptide of the invention comprises an N-terminal truncation at residues 1 to 2 at an equivalent region of SEQ ID NO: 1.

In certain embodiments, the IL-38 polypeptide does not comprise any N-terminal truncation.

Any polypeptide of the invention described herein may also exhibit modified anti-inflammatory properties compared to a polypeptide having an amino acid sequence of SEQ ID NO: 1, an IL-38 amino acid sequence that is unmodified or unmutated, or a IL-38 polypeptide that does not exhibit a reduced capacity to form a dimer. Preferably the modification in anti-inflammatory properties is an increase in anti-inflammatory activity, where anti-inflammatory properties are understood to include effects on the innate as well as the adaptive arms of the immune system. The anti-inflammatory properties or activities may be determined by an assay described herein, particularly in the Examples.

In another aspect, the present invention also provides a fusion protein comprising a polypeptide of the invention as described herein and an Fc region of an antibody. Preferably, the polypeptide has a mutation of one or more cysteine residues at, or at a position equivalent to, 37, 38, 43, 67, 70 and 123 of SEQ ID NO: 1 or a functional variant thereof. In one embodiment, the polypeptide has an amino acid sequence of SEQ ID NO:4 or 5, preferably wherein a substitution of one or more cysteine residues at, or at a position equivalent to, 35, 36, 41, 65, 68 and 121 of SEQ ID NO 4 or 5, or a functional variant thereof. (Ie the skilled person will appreciate that residues 37, 38, 43, 67, 70 and 123 according to the numbering of SEQ ID NO: 1 and 2 are at positions 35, 36, 41, 65, 68 and 121 of SEQ ID NOs: 4 and 5).

In any aspect, the polypeptide in a fusion protein of the invention comprises:
i) an amino acid residue selected from the group consisting of serine, threonine, glycine, alanine, valine, arginine, glutamine and asparagine, optionally serine, at, or at position equivalent to, 37 of SEQ ID NO: 1;
ii) an amino acid residue selected from the group consisting of serine, threonine, glycine, alanine, valine, arginine, glutamine and asparagine, optionally serine, at, or at position equivalent to, 38 of SEQ ID NO: 1;
iii) an amino acid residue selected from the group consisting of serine, threonine, glycine, alanine, valine, arginine, glutamine and asparagine, optionally serine, at, or at position equivalent to, 43 of SEQ ID NO: 1;
iv) an amino acid residue selected from the group consisting of serine, threonine, glycine, alanine, valine, arginine, glutamine and asparagine, optionally serine, at, or at position equivalent to, 67 of SEQ ID NO: 1;
v) an amino acid residue selected from the group consisting of serine, threonine, glycine, alanine, valine, arginine, glutamine and asparagine, optionally serine, at, or at position equivalent to, 70 of SEQ ID NO: 1; and/or
vi) an amino acid residue selected from the group consisting of serine, threonine, glycine, alanine, valine, arginine, glutamine and asparagine, optionally serine, at, or at position equivalent to, 123 of SEQ ID NO: 1.

In certain embodiments, the polypeptide comprises a serine at, or at a position equivalent to, residues 37, 38, 43, 67, 70 and 123 of SEQ ID NO: 1.

In preferred embodiments, the polypeptide comprises a substitution of at least two, at least three, most preferably at least 4, at least 5 or all of the cysteine at residues 37, 38, 43, 67, 70 and 123 of SEQ ID NO: 1. In particular, the polypeptide preferably comprises a substitution of at least the cysteine at residues 37 and 70 of SEQ ID NO: 1, especially substitution of at least the cysteine at residues 37, 43, 67 and 70; or 37, 38, 67 and 70; or 37, 67, 70 and 123, most especially substitution of the cysteine at residues 37, 38, 70 and 123 or 37, 43, 67 and 70.

In certain embodiments, the polypeptide comprises a substitution of the cysteine at residues 37, 43, 67, 70 and 123, or 37, 38, 67, 70 and 123, or 37, 38, 43, 70 and 123; or 37, 38, 43, 67, and 70.

In particularly preferred embodiments, the substitution of the cysteine residues at positions 37, 38 and/or 123 comprises a substitution to a hydrophilic amino acid residue (such as arginine, lysine, histidine, aspartic acid, glutamic acid, serine, threonine, tyrosine, asparagine or glutamine). According to any embodiment described above, the substitution of the cysteine residue at positions 37, 38 and/or 123 is preferably a substitution to a serine or arginine residue. In especially preferred embodiments, the substitution at residue 37 and/or residue 123 is to a serine residue, and/or the substitution at residue 38 is to an arginine.

In preferred embodiments, the substitution of the cysteine residues at positions 43, 67 and 70 comprises a substitution to a hydrophobic amino acid residue (such as valine, leucine, isoleucine, phenylalanine, methionine, glycine or alanine). According to any embodiment described above, the substitution of the cysteine residue at positions 43, 67 and/or 70 is a substitution to an alanine or valine residue. In especially preferred embodiments, the substitution at residue 43 and/or 67 is to an alanine residue and the substitution at residue 70 is to a valine.

In any aspect, the polypeptide in a fusion protein of the invention comprises:
i) a serine residue at, or at position equivalent to residue 37 of SEQ ID NO: 1;
ii) an arginine residue at, or at position equivalent to residue 38 of SEQ ID NO: 1;
iii) an alanine residue at, or at position equivalent to residue 43 of SEQ ID NO: 1;
iv) an alanine residue at, or at position equivalent to residue 67 of SEQ ID NO: 1;
v) a valine residue at, or at position equivalent to residue 70 of SEQ ID NO: 1; and/or
vi) a serine residue at, or at position equivalent to residue 123 of SEQ ID NO: 1.

In an especially preferred embodiment, the polypeptide comprises the amino acid substitutions C70V, C37S, C38R, C43A, C67A, and C123S (numbered according to SEQ ID NO: 1).

In any aspect, the fusion protein is an anti-inflammatory fusion protein.

In any aspect, the amino acid sequence of polypeptide of the fusion protein comprises a sequence as set forth in any one of SEQ ID NO: 1 to 20. Preferably, the polypeptide comprises or consists of the sequence as set forth in any one of SEQ ID NO: 18 or 19.

In any aspect, the Fc region of the antibody of the fusion protein is an Fc region of an IgG, more preferably IgG1.

In any aspect, the IL-38 polypeptide of the fusion protein may be fused at the C-terminus to the Fc region. Alternatively, the IL-38 polypeptide of the fusion protein may be fused via a linker at the C-terminus to the Fc region.

Preferably, the Fc region of the fusion protein comprises two heavy chain fragments, more preferably the CH2 and CH3 domains of said heavy chain.

In preferred embodiments of the invention, the fusion protein comprises a single IL-38 chain, such that the fusion comprises an IL-38 polypeptide as herein described, wherein the Fc portion of the fusion protein is incapable of homodimerisation. As such, the fusion proteins of the invention are preferably those which are capable of being monomeric in the context of IL-38.

The skilled person will be familiar with technology and Fc sequences for enabling the formation of so-called monomeric fusion proteins, including although not limited to the use of the “knobs-into-holes” IgG1 format. Such approaches in the context of the present invention, enable expression and purification of an IL-38-Fc with only one copy of IL-38 per molecule (for example as depicted in FIG. 24A; and in contrast to convention Fc-fusion proteins which comprise identical fusion-Fc chains that dimerise and therefore comprise two copies of the moiety that is fused to the Fc). One example of a “knob-into-hole” Fc pairing is provided in SEQ ID NO: 20 (Fc knob) and SEQ ID NO: 9 (Fc hole). An example of this arrangement in the context of s fusion protein of the invention is also provided in SEQ ID NOs: 18, 19 and 21, wherein the IL-38 polypeptide is fused to an IgG1 “hole” Fc sequence.

As such, a fusion protein of the invention is preferably one that is capable of forming a heterodimeric molecule that comprises a single IL-38 polypeptide amino acid sequence. (In other words, the Fc portion of the fusion protein may form a heterodimer with an Fc region of an antibody that does not comprise an IL-38 polypeptide fused thereto).

In another aspect, the invention provides a pharmaceutical composition for treating or preventing an inflammatory disease or condition comprising a polypeptide or fusion protein of the invention and a pharmaceutically acceptable diluent, excipient or carrier. In one embodiment, the only active ingredient present in the composition is a polypeptide of the invention.

In any aspect of the present invention, 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% of the polypeptide of the invention present in a pharmaceutical composition of the invention is monomeric. Alternatively, less than 50%, 40%, 30%, 25%, 19%, 18%, 17%, 16%, 15%, 14%, 13%, 12%, 11%, 10%, 9%, 8%, 7%, 6%, 5%, 4%, 3%, 2% or 1% the polypeptide of the invention present in a pharmaceutical composition of the invention is homodimeric.

In any aspect of the present invention, 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% of the polypeptide of the invention, particularly an IL-38 fusion protein of the invention, present in a pharmaceutical composition of the invention, is in a heterodimeric conformation. Alternatively, less than 50%, 40%, 30%, 25%, 19%, 18%, 17%, 16%, 15%, 14%, 13%, 12%, 11%, 10%, 9%, 8%, 7%, 6%, 5%, 4%, 3%, 2% or 1% the polypeptide of the invention present in a pharmaceutical composition of the invention is homodimeric.

In another aspect, the invention provides a pharmaceutical composition for treating or preventing an inflammatory disease or condition comprising as an active ingredient a polypeptide or fusion protein of the invention and a pharmaceutically acceptable diluent, excipient or carrier. In one embodiment, the only active ingredient present in the composition is a polypeptide or fusion protein of the invention.

In another aspect, the invention provides a pharmaceutical composition for treating or preventing an inflammatory disease or condition comprising as a main ingredient a polypeptide or fusion protein of the invention and a pharmaceutically acceptable diluent, excipient or carrier. In one embodiment, the only active ingredient present in the composition is a polypeptide of the invention.

In another aspect, the invention also provides a polypeptide or fusion protein of the invention for use in the treatment of an inflammatory disease or condition.

In another aspect, the invention also provides a pharmaceutical composition comprising a polypeptide or fusion protein of the invention and a pharmaceutically acceptable diluent, excipient or carrier for use in the treatment of an inflammatory disease or condition.

In another aspect, the invention also provides a method of inhibiting inflammation in a subject in need thereof, the method comprising administering to the subject a therapeutically effective amount of a polypeptide or fusion protein of the invention, or a pharmaceutical composition of the invention, thereby inhibiting inflammation in the subject.

In another aspect, the present invention provides a method for the treatment or prevention of an inflammatory disease or condition, the method comprising the step of administering a composition to the subject for treatment or prevention, wherein the composition comprises, consists essentially of or consists of a polypeptide or fusion protein of the invention and a pharmaceutically acceptable diluent, excipient or carrier.

In any method or use of the invention described herein, a polypeptide or fusion protein, or pharmaceutical composition of the invention may be administered systemically or directly to the site of disease. A polypeptide, fusion protein or pharmaceutical composition of the invention may be formulated for oral administration.

In another aspect, the present invention provides a method of treating or preventing an inflammatory disease or condition in a subject in need thereof, the method comprising administering to the subject a therapeutically effective amount a polypeptide, fusion protein, or pharmaceutical composition of the invention, thereby treating or preventing an inflammatory disease or condition in a subject.

In another aspect, the invention also provides a method of alleviating or ameliorating a symptom of an inflammatory disease or condition in a subject in need thereof, the method comprising administering to the subject in need thereof a therapeutically effective amount of a polypeptide, fusion protein or pharmaceutical composition of the invention, thereby alleviating or ameliorating a symptom of an inflammatory disease or condition in the subject.

In another aspect, the invention also provides use of a therapeutically effective amount of a polypeptide, fusion protein or pharmaceutical composition of the invention in the manufacture of a medicament for the treatment or prevention of an inflammatory disease or condition in a subject in need thereof.

In another aspect, the present invention provides a method for the treatment of an inflammatory disease or condition in a subject comprising the steps of:
identifying a subject having an inflammatory disease or condition; and
administering to the subject in need thereof a therapeutically effective amount of a polypeptide, fusion protein or pharmaceutical composition of the invention, thereby treating an inflammatory disease or condition in the subject.

In another aspect, the invention also provides a nucleic acid molecule encoding a polypeptide or fusion protein as described herein.

In another aspect, the invention also provides a vector comprising a nucleic acid molecule described herein.

In another aspect, the invention also provides a cell comprising a vector or nucleic acid molecule described herein.

In another aspect, the invention also provides an animal or tissue derived therefrom comprising a cell described herein.

As used herein, except where the context requires otherwise, the term “comprise” and variations of the term, such as “comprising”, “comprises” and “comprised”, are not intended to exclude further additives, components, integers or steps. The term “including” is also used interchangeably with “comprising” and is also not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: IL-38 construct design. (A) Vector map of pET22b(+) IL-38, used for the generation of IL-38 for use in crystallographic analysis. IL-38 was commercially synthesised (Genscript) and cloned into pET22b+ (Novagen) for bacterial expression using a T7 promoter. A Histidine tag (His₁₀) was added to the IL-38 N-terminus to enable affinity purification and a TEV protease site was added downstream of the His₁₀ tag to enable removal during purification. (B) The final IL-38 coding sequence used for all subsequent experiments unless otherwise noted. The sequence accession code is NP_115945. Common gene/protein names associated with this sequence include ILIF10, FIL1T, IL1HY2, IL38, FKSG75, and UNQ6119/PRO20041.

FIG. 2: IL-38 purification. (A) IL-38 was expressed in BL21-CodonPlus(DE3)-RIL cells (Stratagene) by IPTG induction (OD₆₀₀ 0.8) overnight at 18° C. The cells were lysed by sonication and the soluble fraction was bound to Ni-NTA resin (Qiagen). After nickel elution, IL-38 was highly pure (Lane 1) and subsequent overnight Tobacco Etch Virus (TEV) cleavage resulted in removal of the His₁₀ tag from approximately 50% of the protein (Lane 2). (B) The eluted protein was loaded onto a 1 ml HiTrapQ column and eluted with a NaCl gradient from 0.05M to 1M NaCl. The eluted protein was analysed by SDS-PAGE and contained both cleaved and uncleaved IL-38 (Lanes 1-3). (C) IL-38 was loaded onto a Superdex75 16/60 (Cytiva Life Sciences) column and eluted in two peaks with an elution volume indicated a size corresponding to monomeric and dimeric IL-38 conformations. (D) Uncut IL-38 was removed from the Peak A protein pool by further incubation with Ni-NTA resin and the untagged IL-38 was further purified by SEC. (E) SDS-PAGE analysis of SEC run shown in (D).

FIG. 3: Analytical SEC analysis of IL-38. (A) Purified IL-38 from Peak A (orange) and Peak B (blue) was analysed on an analytical Superdex 75 10/300 GL column (Cytiva Life Sciences) to give an indication of the size of the two protein conformations. Monomeric recombinant IL-362 was loaded onto the column as a control. The IL-38 Peak A eluted off the column at a volume indicative of a homodimer and IL-38 Peak B eluted off the column at a volume indicative of a monomer. Importantly, neither Peak A nor Peak B of IL-38 readily interconverted during storage or SEC. These data indicate that Peak A and Peak B are not in equilibrium. A traditional head-to-head homodimer (e.g. IL-37) would be expected to dissociate back into a monomer, especially at concentrations whereby both monomer and dimer are observed during purification.

FIG. 4: SEC and multi-angle light scattering analysis (SEC-MALS) of the IL-38 dimer. Purified IL-38 dimer was analysed by SEC-MALS on a Superdex 75 10/300 GL column (Cytiva Life Sciences). The IL-38 homodimer elutes as a single peak by SEC (left axis-absorbance 280 nm) with a calculated molecular weight of between 30000-32000 Daltons (right axis, multi-angle light scatter calculation of molecular weight).

FIG. 5: IL-38 monomer-dimer structure. The crystal structure of the homodimer was solved to 3.4 Å. It was determined that IL-38 forms a domain swapped dimer. The β-trefoil fold domain swaps at the β4-β5 loop.

FIG. 6: IL-38 homodimer hinge region. Superposition of the IL-38 monomer (grey; 5BOW) with the IL-38 homodimer (blue) highlighting the hinge region. The domain-swap hinge region is formed by the loop between strands β4 and β5. Pro46 and Gly49 are shown as sticks.

FIG. 7: IL-38 monomer loop. The IL-38 loop contains two structural motifs including an Asx turn whereby Asp51 forms a hydrogen bond to the main chain nitrogen of Thr53. A nest motif is also formed Gly49, Leu50, and Asp51. Nest motifs can form binding sites for an anionic group (termed the egg). As such anions may stabilise the loop conformation (e.g. phosphates, iron-sulphur clusters).

FIG. 8: IL-38 SNP. (A) Comparison of the IL-38 monomeric and dimeric structures reveals the mechanism of dimer formation. The IL-38 monomer structure is of a SNP (rs6743376) with a mutation at residue 51 (A51D). (B) & (C) The solved IL-38 homodimer structure is the WT (D51A) sequence with an alanine at position 51. Aspartic acid at position 51 stabilises the β-turn reducing dimer formation.

FIG. 9: Monomer/homodimer. WT IL-38 forms ˜20% dimer upon purification.

FIG. 10: Stability of WT IL-38. WT IL-38 exists in a slow equilibrium between the two states. IL-38 (A51D) is a stable monomer.

FIG. 11: IL-38 mutations. The homodimer interface can be can modulated and form a stable locked dimer (ΔT53). Various other mutations potentiate or hinder homodimer formation.

FIG. 12: Locked homodimer. The locked dimer (ΔT53) variant is stable as a dimer (e.g. versus G49P upon prolonged incubation at 37 deg for 16 hrs).

FIG. 13: Expression of IL-38 fusions. IL-38 (WT) or IL-38 A51D expression containing unpaired cysteines or with cysteines removed by mutation to serine. Top arrow indicating IL-38-Fc fusion, bottom arrow indicating unfused Fc. Removal of cysteines from IL-38 (WT) enables expression of the Fc fusion. IL-38 (A51D) can be produced with cysteines present but with reduced expression.

FIG. 14: Stability of IL-38 fusions. Removal of cysteines from IL-38 (A51D) increases yield and stability of the Fc fusion.

FIG. 15: Linker length. IL-38-Fc fusions were generated with different linker lengths (e.g. 6 amino acids vs 22 amino acids (GS-based linker)).

FIG. 16: Fc fusion minimises dimer formation. Compared to the protein alone (A) the addition of an Fc moiety minimises IL-38 dimerization (B).

FIG. 17: IL-38-Fc reduces Poly (I:C)-induced IRF3/7 Quanti-Luc in Human NF-kB-SEAP & IRFLuc Reporter A549-Dual cells from Invivogen (lung epithelial cells). A549-Dual cells were pretreated for 1 h with lipofectamine and (1 ng/ml) Poly (I:C) low molecular weight (LMW) and then stimulated dose dependently (A) 100 nM/l, (B) 1 nM/l, (C) 10 pM/l, (D) 100 fM/l with different IL-38 variants or dexamethasone (5 nM) as benchmark for 20 h. Depicted is the suppression of Quanti-Luc by IL-38 variants in % change to Poly (I:C) (mean+/−SEM), n>10 experiments carried out in triplicate. *, P<0.05, **, P<0.01, ***, P<0.001 and ****, P<0.0001 for Poly (I:C) alone vs Poly (I:C)+IL-38 variant treatment. o, P<0.05, 00, P<0.01, 000, P<0.001 and oooo, P<0.0001 for Poly (I:C)+variants vs Poly (I:C)+dT53 treatment. #, P <0.05, ##P<0.01, ## #, P<0.001 and ## ##, P<0.0001 for Poly (I:C)+variants vs Poly (I:C)+A51D treatment. ns: not significant.

FIG. 18: IL-38-Fc reduces Poly (I:C)-induced IRF3/7 Quanti-Luc in Human NF-kB-SEAP & IRFLuc Reporter A549-Dual cells from Invivogen (lung epithelial cells). Different graphing of data of FIG. 15, statistics on comparison of different dose response curves. * A51D vs D51A Fc 6aa, *** A51D vs dT53, ns A51D vs D51A, ns A51D vs A51D Fc 6aa, dT53 vs D51A Fc 6aa, ns dT53 vs D51A, ** dT53 vs A51 D Fc 6aa, * D51A vs D51A Fc 6aa, ns D51A vs A51D Fc 6aa, ** A51D Fc 6aa vs D51A Fc 6aa.

FIG. 19: IL-38-Fc reduces Poly (I:C)-induced RANTES and IL-6 protein expression and IFNL, IFNB, ISG15 and CXCL10 mRNA expression levels in A549-Dual cells from Invivogen (lung epithelial cells). A549-Dual cells were pretreated for 1 h with lipofectamine and (1 ng/ml) Poly (I:C) low molecular weight (LMW) and then stimulated with 1 nM/l of different IL-38 variants or dexamethasone (5 nM) as benchmark for 20 h. Depicted is the suppression of protein expression of (A) RANTES, or (B) IL-6 and mRNA expression levels for (C) IFNL, (D) IFNB, (E) ISG15 and (F) CXCL10 by IL-38 variants in % change to Poly (I:C) (mean+/−SEM), n>10 experiments carried out in triplicate. *, P<0.05, **, P<0.01, ***, P<0.001 and ****, P<0.0001 for Poly (I:C) alone vs Poly (I:C)+IL-38 variant treatment. o, P<0.05, 00, P<0.01, 000, P<0.001 and oooo, P<0.0001 for Poly (I:C)+variants vs Poly (I:C)+dT53 treatment. #, P<0.05, ##P<0.01, ## #, P<0.001 and ## ##, P<0.0001 for Poly (I:C)+variants vs Poly (I:C)+A51D treatment. \hat{ }, P<0.05, \hat{ }\hat{ }P<0.01, \hat{ }\hat{ }\hat{ }, P<0.001 and \hat{ }\hat{ }\hat{ }\hat{ }, P<0.0001 for Poly (I:C)+variants Poly (I:C)+dexamethasone treatment ns: not significant.

FIG. 20: Functional testing of fusion proteins in mechanistic model for disease relevant pathway TLR7 stimulation. IL-38 D51A-Fc presents with activity in a mechanistic murine model of disease (imiquimod (imi) 5 μg/g intraperitoneal (i.p.). S.c. administration of D51A-Fc and A51D Fc-fusion was tested at 40 μg/kg normalized to molecular weight 1h prior to imiquimod (i.p.) injections. Serum and spleens were harvested at 6h after imiquimod injection and IFNalpha, IFNbeta and IL-6 were determined by ELISA. Readouts in spleenlysates were normalized to total protein. Depicted are two independent experiments, n=5-10/group.

FIG. 21: Size-exclusion chromatography analysis of IL-38-Fc cysteine variants. A-F. The quality of IL-38-Fc variants was monitored by size exclusion chromatography. A representative successful purification of the “deltaCys” IL-38-Fc (C70V, C37S, C38R, C43A, C67A, C123S) is displayed in each panel with an elution profile of a single peak corresponding to a homogenous preparation of pure IL-38-Fc. A-F. Select indicated variants are displayed to demonstrate the increase in IL-38-Fc purification quality upon sequential mutation of cysteine residues within the IL-38 moiety.

FIG. 22. Inhibitory effect of IL-38-Fc truncation variants on Poly (I:C)-induced IRF3/7 Quanti-Luc in human NF-kB-SEAP & IRFLuc Reporter A549-Dual cells from Invivogen. A549-Dual cells were dose-dependently pre-treated for 2 h with different D51A 6DCys IL-38 Fc fusions. Thereafter cells were stimulated for 1 h with lipofectamine and (1 ng/ml) Poly (I:C) low molecular weight (LMW), followed by a media change. Depicted is the suppression of Quanti-Luc at 20 hours by IL-38 Fc variants in % change to Poly (I:C)+Fc control (mean+/−SEM), n>10 experiments carried out in triplicate.

FIG. 23 Inhibitory effect of IL-38-Fc variants on Poly (I:C)-induced IRF3/7 Quanti-Luc or RANTES in Human NF-kB-SEAP & IRFLuc Reporter A549-Dual cells from Invivogen. A549-Dual cells were dose-dependently pre-treated for 2 h with different D51A 6ΔCys IL-38 Fc fusions, dexamethasone (5 nM) or anifrolumab (10 pm) as benchmark. Thereafter cells were stimulated for 1 h with lipofectamine and (1 ng/ml) Poly (I:C) low molecular weight (LMW), followed by a media change. Depicted is the suppression of RANTES by AlphaLISA (FIG. 3 B) or Quanti-Luc (FIGS. 3A, C and D) at 20 hours by IL-38 Fc variants, dexamethasone or anifrolumab in % change to Poly (I:C)+Fc control (mean+/−SEM), n>10 experiments carried out in triplicate.

FIG. 24. Comparison of IL-38 in a knobs-into-holes IgG1 Fc format (one IL-38 copy) or a standard IgG1 (two IL-38 copies). A. SDS-PAGE analysis of purified IL-38-Fc in a knobs-into-holes format. The IL-38-Fc knob chain is larger and migrates on the SDS-PAGE at ˜50 kDa with the Fc-hole chain migrating at ˜30 kDa. A schematic representation of IL-38-Fc in a knobs-into-holes format. B. SDS-PAGE analysis of purified IL-38-Fc in a standard IgG₁ format. Each IL-38-Fc chain migrate by SDS-PAGE at ˜50 kDa. A schematic representation of IL-38-Fc in a standard IgG₁ format. C. Size exclusion chromatography of IL-38-Fc with one IL-38 copy in comparison to IL-38-Fc with two IL-38 copies. D. IRF3/7Luc Reporter A549-Dual cells from Invivogen were pre-treated for 2 h with 1 nM of either a “knobs-into-holes” or standard IgG1 non KiH D51A ΔCys IL-38 Fc fusion. Thereafter cells were stimulated with lipofectamine and (1 ng/ml) Poly (I:C) low molecular weight (LMW) for 1h followed by a media change. Depicted is the suppression of Quanti-Luc by IL-38 Fc variants in % change to Poly (I:C)+Fc control (mean+/−SEM), n>10 experiments carried out in triplicate. *, P<0.05, **, P<0.01, for Poly (I:C)+Fc control vs Poly (I:C)+IL-38 Fc treatment.

TABLE 1
Sequences of the invention

| SEQ ID | | |
|---|---|---|
| NO: | Description | Sequence |
| 1 | Wild-type IL-38 (hinge | MCSLPMARYYIIKYADQKALYTRDGQLLVGDPVAD |
| | region forming domain- | NCCAEKICILPNRGLARTKVPIFLGIQGGSRCLACVET |
| | swap dimerization | EEGPSLQLEDVNIEELYKGGEEATRFTFFQSSSGSAF |
| | interface underlined) | RLEAAAWPGWFLCGPAEPQQPVQLTKESEPSARTKF |
| | Residues 1-152 | YFEQSW |
| 2 | Variant of IL-38 that | MCSLPMARYYIIKYADQKALYTRDGQLLVGDPVAD |
| | forms monomer | NCCAEKICILPNRGLDRTKVPIFLGIQGGSRCLACVET |
| | Residues 1-152 | EEGPSLQLEDVNIEELYKGGEEATRFTFFQSSSGSAF |
| | | RLEAAAWPGWFLCGPAEPQQPVQLTKESEPSARTKF |
| | | YFEQSW |
| 3 | Variant sequence at | MSSSFLPEPLPAKSLQHGVPLSLDSSLSSLL |
| | equivalent position to | |
| | residues 9 to 39 of SEQ | |
| | ID NO: 1 (Isoform 2) | |
| 4 | Wild-type IL-38 | SLPMARYYIIKYADQKALYTRDGQLLVGDPVADNC |
| | Residues 3-152 | CAEKICILPNRGLARTKVPIFLGIQGGSRCLACVETEE |
| | | GPSLQLEDVNIEELYKGGEEATRFTFFQSSSGSAFRL |
| | | EAAAWPGWFLCGPAEPQQPVQLTKESEPSARTKFYF |
| | | EQSW |
| 5 | Variant of IL-38 that | SLPMARYYIIKYADQKALYTRDGQLLVGDPVADNC |
| | forms monomer | CAEKICILPNRGLDRTKVPIFLGIQGGSRCLACVETEE |
| | Residues 3-152 | GPSLQLEDVNIEELYKGGEEATRFTFFQSSSGSAFRL |
| | | EAAAWPGWFLCGPAEPQQPVQLTKESEPSARTKFYF |
| | | EQSW |
| 6 | Variant of IL-38 T53N | SLPMARYYIIKYADQKALYTRDGQLLVGDPVADNC |
| | with increased capacity | CAEKICILPNRGLARNKVPIFLGIQGGSRCLACVETEE |
| | to form monomer | GPSLQLEDVNIEELYKGGEEATRFTFFQSSSGSAFRL |
| | Residues 3-152 | EAAAWPGWFLCGPAEPQQPVQLTKESEPSARTKFYF |
| | | EQSW |
| 7 | Variant of IL-38 V55T | SLPMARYYIIKYADQKALYTRDGQLLVGDPVADNC |
| | with increased capacity | CAEKICILPNRGLARTKTPIFLGIQGGSRCLACVETEE |
| | to form monomer | GPSLQLEDVNIEELYKGGEEATRFTFFQSSSGSAFRL |
| | Residues 3-152 | EAAAWPGWFLCGPAEPQQPVQLTKESEPSARTKFYF |
| | | EQSW |
| 8 | Variant of IL-38 V55Q | SLPMARYYIIKYADQKALYTRDGQLLVGDPVADNC |
| | with increased capacity | CAEKICILPNRGLARTKQPIFLGIQGGSRCLACVETEE |
| | to form monomer | GPSLQLEDVNIEELYKGGEEATRFTFFQSSSGSAFRL |
| | Residues 3-152 | EAAAWPGWFLCGPAEPQQPVQLTKESEPSARTKFYF |
| | | EQSW |
| 10 | N-terminally truncated | MARYYIIKYADQKALYTRDGQLLVGDPVADNCCAE |
| | variation of wild-type IL- | KICILPNRGLARTKVPIFLGIQGGSRCLACVETEEGPS |
| | 38 | LQLEDVNIEELYKGGEEATRFTFFQSSSGSAFRLEAA |
| | Residues 6-152 | AWPGWFLCGPAEPQQPVQLTKESEPSARTKFYFEQS |
| | | W |
| 11 | N-terminally truncated | MARYYIIKYADQKALYTRDGQLLVGDPVADNCCAE |
| | variant of IL-38 that | KICILPNRGLDRTKVPIFLGIQGGSRCLACVETEEGPS |
| | forms monomer | LQLEDVNIEELYKGGEEATRFTFFQSSSGSAFRLEAA |
| | Residues 6-152 | AWPGWFLCGPAEPQQPVQLTKESEPSARTKFYFEQS |
| | | W |
| 12 | N-terminally truncated | LYTRDGQLLVGDPVADNCCAEKICILPNRGLDRTKV |
| | variant of IL-38 that | PIFLGIQGGSRCLACVETEEGPSLQLEDVNIEELYKG |
| | forms monomer | GEEATRFTFFQSSSGSAFRLEAAAWPGWFLCGPAEP |
| | Residues 20-152 | QQPVQLTKESEPSARTKFYFEQSW |
| 13 | Variant of IL-38 with | SLPMARYYIIKYADQKALYTRDGQLLVGDPVADNC |
| | increased capacity to | SAEKICILPNRGLDRTKVPIFLGIQGGSRCLACVETEE |
| | form monomer; and | GPSLQLEDVNIEELYKGGEEATRFTFFQSSSGSAFRL |
| | suitable for conjugation | EAAAWPGWFLSGPAEPQQPVQLTKESEPSARTKFYF |
| | to Fc | EQSW |
| | Residues 3-152; A51D | |
| | C38S C123S | |
| 14 | Variant of IL-38 with | SLPMARYYIIKYADQKALYTRDGQLLVGDPVADNSS |
| | increased capacity to | AEKICILPNRGLDRTKVPIFLGIQGGSRCLACVETEEG |
| | form monomer; and | PSLQLEDVNIEELYKGGEEATRFTFFQSSSGSAFRLE |
| | suitable for conjugation | AAAWPGWFLSGPAEPQQPVQLTKESEPSARTKFYFE |
| | to Fc | QSW |
| | Residues 3-152; A51D | |
| | C37S C38S C123S | |
| 15 | Variant of IL-38 with | SLPMARYYIIKYADQKALYTRDGQLLVGDPVADNSS |
| | increased capacity to | AEKICILPNRGLDRTKVPIFLGIQGGSRCLACVETEEG |
| | form monomer; and | PSLQLEDVNIEELYKGGEEATRFTFFQSSSGSAFRLE |
| | suitable for conjugation | AAAWPGWFLSGPAEPQQPVQLTKESEPSARTKFYFE |
| | to Fc | QSW |
| | Residues 3-152; A51D | |
| | C43S C38S C123S | |
| 16 | Variant of IL-38 with | SLPMARYYIIKYADQKALYTRDGQLLVGDPVADNSS |
| | increased capacity to | AEKISILPNRGLDRTKVPIFLGIQGGSRCLACVETEEG |
| | form monomer; and | PSLQLEDVNIEELYKGGEEATRFTFFQSSSGSAFRLE |
| | suitable for conjugation | AAAWPGWFLSGPAEPQQPVQLTKESEPSARTKFYFE |
| | to Fc | QSW |
| | Residues 3-152; A51D | |
| | C37S C43S C38S | |
| 17 | Variant of IL-38 with | SLPMARYYIIKYADQKALYTRDGQLLVGDPVADNSS |
| | increased capacity to | AEKIAILPNRGLDRTKVPIFLGIQGGSRCLACVETEEG |
| | form monomer; and | PSLQLEDVNIEELYKGGEEATRFTFFQSSSGSAFRLE |
| | suitable for conjugation | AAAWPGWFLSGPAEPQQPVQLTKESEPSARTKFYFE |
| | to Fc | QSW |
| | Residues 3-152; A51D | |
| | C37S C38S C43S C123S | |
| 18 | IL-38 with modified Cys | SLPMARYYIIKYADQKALYTRDGQLLVGDPVADNS |
| | residues + Hole Fc | RAEKIAILPNRGLARTKVPIFLGIQGGSRALAVVETEE |
| | (underlined) | GPSLQLEDVNIEELYKGGEEATRFTFFQSSSGSAFRL |
| | | EAAAWPGWFLSGPAEPQQPVQLTKESEPSARTKFYF |
| | | EQSWGSGSGSDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALPAPIEKTISKAKGQPREPQVYTLPPC |
| | | RDELTKNQVSLWCLVKGFYPSDIAVEWESNGQPEN |
| | | NYKTTPPVLDSDGSFFLYSKLTVDKSRWQQGNVFSC |
| | | SVMHEALHNHYTQKSLSLSPGK |
| 19 | IL-38 that forms a dimer | SLPMARYYIIKYADQKALYTRDGQLLVGDPVADNS |
| | and with modified Cys | RAEKIAILPNRGLDRTKVPIFLGIQGGSRALAVVETEE |
| | residues + Hole Fc | GPSLQLEDVNIEELYKGGEEATRFTFFQSSSGSAFRL |
| | (underlined) | EAAAWPGWFLSGPAEPQQPVQLTKESEPSARTKFYF |
| | | EQSWGSGSGSDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALPAPIEKTISKAKGQPREPQVYTLPPC |
| | | RDELTKNQVSLWCLVKGFYPSDIAVEWESNGQPEN |
| | | NYKTTPPVLDSDGSFFLYSKLTVDKSRWQQGNVESC |
| | | SVMHEALHNHYTQKSLSLSPGK |
| 20 | Knob Fc | DKTHTCPPCPAPEAAGGPSVFLFPPKPKDTLMISRTP |
| | | EVTCVVVDVSHEDPEVKFNWYVDGVEVHNAKTKP |
| | | REEQYNSTYRVVSVLTVLHQDWLNGKEYKCKVSN |
| | | KALPAPIEKTISKAKGQPREPQVCTLPPSRDELTKNQ |
| | | VSLSCAVKGFYPSDIAVEWESNGQPENNYKTTPPVL |
| | | DSDGSFFLVSKLTVDKSRWQQGNVFSCSVMHEALH |
| | | NRFTQKSLSLSPGK |
| 9 | Hole Fc | DKTHTCPPCPAPEAAGGPSVFLFPPKPKDTLMISRTP |
| | | EVTCVVVDVSHEDPEVKFNWYVDGVEVHNAKTKP |
| | | REEQYNSTYRVVSVLTVLHQDWLNGKEYKCKVSN |
| | | KALPAPIEKTISKAKGQPREPQVYTLPPCRDELTKNQ |
| | | VSLWCLVKGFYPSDIAVEWESNGQPENNYKTTPPVL |
| | | DSDGSFFLYSKLTVDKSRWQQGNVESCSVMHEALH |
| | | NHYTQKSLSLSPGK |
| 21 | “Knobs-into-holes” IgG1 | MGWSCIILFLVATATGVHSSLPMARYYIIKYADQKAL |
| | IL-38-Fc residues 3-152 | YTRDGQLLVGDPVADNSRAEKIAILPNRGLARTKVPI |
| | (C70V, C37S, C38R, | FLGIQGGSRALAVVETEEGPSLQLEDVNIEELYKGGE |
| | C43A, C67A, C123S). | EATRFTFFQSSSGSAFRLEAAAWPGWFLSGPAEPQQ |
| | GSGSGS linker. Human | PVQLTKESEPSARTKFYFEQSWGSGSGSDKTHTCPPC |
| | IgG2 heavy chain leader | PAPEAAGGPSVFLFPPKPKDTLMISRTPEVTCVVVDV |
| | sequence in italics | SHEDPEVKFNWYVDGVEVHNAKTKPREEQYNSTYR |
| | Chain A | VVSVLTVLHQDWLNGKEYKCKVSNKALPAPIEKTIS |
| | | KAKGQPREPQVYTLPPCRDELTKNQVSLWCLVKGF |
| | | YPSDIAVEWESNGQPENNYKTTPPVLDSDGSFFLYS |
| | | KLTVDKSRWQQGNVFSCSVMHEALHNHYTQKSLSL |
| | | SPGK |
| 22 | Chain B IgG1 sequence | MGWSCIILFLVATATGVHSDKTHTCPPCPAPEAAGGPS |
| | for use with “Knobs-into- | VFLFPPKPKDTLMISRTPEVTCVVVDVSHEDPEVKFN |
| | holes” IgG1 IL-38-Fc | WYVDGVEVHNAKTKPREEQYNSTYRVVSVLTVLH |
| | residues 3-152 (C70V, | QDWLNGKEYKCKVSNKALGAPIEKTISKAKGQPREP |
| | C37S, C38R, C43A, | QVCTLPPSRD |
| | C67A, C123S). | ELTKNQVSLSCAVKGFYPSDIAVEWESNGQPENNYK |
| | GSGSGS linker. Human | TTPPVLDSDGSFFLVSKLTVDKSRWQQGNVFSCSVM |
| | IgG2 heavy chain leader | HEALHNRFTQKSLSLSPGK |
| | sequence in italics | |
| 23 | IgG1 IL-38-Fc, residues | MGWSCIILFLVATATGVHSSLPMARYYIIKYADQKAL |
| | 3-152 (C70V, C37S, | YTRDGQLLVGDPVADNSRAEKIAILPNRGLARTKVPI |
| | C38R, C43A, C67A, | FLGIQGGSRALAVVETEEGPSLQLEDVNIEELYKGGE |
| | C123S). No linker. | EATRFTFFQSSSGSAFRLEAAAWPGWFLSGPAEPQQ |
| | Human IgG2 heavy chain | PVQLTKESEPSARTKFYFEQSWDKTHTCPPCPAPEA |
| | leader sequence in italics. | AGGPSVFLFPPKPKDTLMISRTPEVTCVVVDVSHEDP |
| | | EVKFNWYVDGVEVHNAKTKPREEQYNSTYRVVSV |
| | | LTVLHQDWLNGKEYKCKVSNKALPAPIEKTISKAKG |
| | | QPREPQVYTLPPSRDELTKNQVSLTCLVKGFYPSDIA |
| | | VEWESNGQPENNYKTTPPVLDSDGSFFLYSKLTVDK |
| | | SRWQQGNVFSCSVMHEALHNHYTQKSLSLSPGK |
| 24 | Variant of IL-38 with | SLPMARYYIIKYADQKALYTRDGQLLVGDPVADNS |
| | increased capacity to | RAEKIAILPNRGLARTKVPIFLGIQGGSRALAVVETEE |
| | form monomer; and | GPSLQLEDVNIEELYKGGEEATRFTFFQSSSGSAFRL |
| | suitable for conjugation | EAAAWPGWFLSGPAEPQQPVQLTKESEPSARTKFYF |
| | to Fc | EQSW |
| | Residues 3-152; A51D | |
| | C70V, C37S, C38R, | |
| | C43A, C67A, C123S | |
| 25 | “Knobs-into-holes” IgG1 | SLPMARYYIIKYADQKALYTRDGQLLVGDPVADNS |
| | IL-38-Fc residues 3-152 | RAEKIAILPNRGLARTKVPIFLGIQGGSRALAVVETEE |
| | (C70V, C37S, C38R, | GPSLQLEDVNIEELYKGGEEATRFTFFQSSSGSAFRL |
| | C43A, C67A, C123S). | EAAAWPGWFLSGPAEPQQPVQLTKESEPSARTKFYF |
| | GSGSGS linker. Human | EQSWGSGSGSDKTHTCPPCPAPEAAGGPSVFLFPPKP |
| | IgG2 heavy chain leader | KDTLMISRTPEVTCVVVDVSHEDPEVKFNWYVDGV |
| | sequence removed | EVHNAKTKPREEQYNSTYRVVSVLTVLHQDWLNGK |
| | Chain A | EYKCKVSNKALPAPIEKTISKAKGQPREPQVYTLPPC |
| | | RDELTKNQVSLWCLVKGFYPSDIAVEWESNGQPEN |
| | | NYKTTPPVLDSDGSFFLYSKLTVDKSRWQQGNVFSC |
| | | SVMHEALHNHYTQKSLSLSPGK |
| 26 | Chain B IgG1 sequence | DKTHTCPPCPAPEAAGGPSVFLFPPKPKDTLMISRTP |
| | for use with “Knobs-into- | EVTCVVVDVSHEDPEVKFNWYVDGVEVHNAKTKP |
| | holes” IgG1 IL-38-Fc | REEQYNSTYRVVSVLTVLHQDWLNGKEYKCKVSN |
| | residues 3-152 (C70V, | KALGAPIEKTISKAKGQPREPQVCTLPPSRD |
| | C37S, C38R, C43A, | ELTKNQVSLSCAVKGFYPSDIAVEWESNGQPENNYK |
| | C67A, C123S). | TTPPVLDSDGSFFLVSKLTVDKSRWQQGNVFSCSVM |
| | GSGSGS linker. Human | HEALHNRFTQKSLSLSPGK |
| | IgG2 heavy chain leader | |
| | sequence removed | |
| 27 | IgG1 IL-38-Fc, residues | SLPMARYYIIKYADQKALYTRDGQLLVGDPVADNS |
| | 3-152 (C70V, C37S, | RAEKIAILPNRGLARTKVPIFLGIQGGSRALAVVETEE |
| | C38R, C43A, C67A, | GPSLQLEDVNIEELYKGGEEATRFTFFQSSSGSAFRL |
| | C123S). No linker. | EAAAWPGWFLSGPAEPQQPVQLTKESEPSARTKFYF |
| | Human IgG2 heavy chain | EQSWDKTHTCPPCPAPEAAGGPSVFLFPPKPKDTLMI |
| | leader sequence removed | SRTPEVTCVVVDVSHEDPEVKFNWYVDGVEVHNAK |
| | | TKPREEQYNSTYRVVSVLTVLHQDWLNGKEYKCKV |
| | | SNKALPAPIEKTISKAKGQPREPQVYTLPPSRDELTK |
| | | NQVSLTCLVKGFYPSDIAVEWESNGQPENNYKTTPP |
| | | VLDSDGSFFLYSKLTVDKSRWQQGNVFSCSVMHEA |
| | | LHNHYTQKSLSLSPGK |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The present invention is based on the surprising finding by the inventors that wild-type IL-38 has the ability to form both monomer and homodimer conformations. In fact, the inventors are the first to identify formation of an IL-38 homodimer.

Moreover, and more surprisingly, the inventors have determined that the manner in which the IL-38 dimer forms is different from the dimerization mechanism observed for other interleukin-type molecules. For example, in IL-37, the dimerization interface is formed by the interaction of two hydrophobic regions in each monomer. The IL-37 dimer is a “head-to-head” dimer, wherein the same region of each protein subunit interacts to form a symmetrical and closed conformation. In contrast, in IL-38, the dimerization interface is alpha-helical and forms a “domain-swap” hinge region. In domain-swapping, regions/domains of each protein subunit swap positions to form a stable dimer. In the case of IL-38, the domain-swap hinge region is formed by the loop between strands β4 and β5 of monomeric IL-38.

Importantly, the inventors have identified that dimerization strongly reduces most functions of IL-38, including its anti-inflammatory properties. Therefore, the present invention is based on this novel and unexpected finding. Accordingly, the present invention is directed to, amongst other things, therapeutic compositions that predominantly comprise monomeric IL-38, various novel mutant IL-38 with reduced capacity to form a dimer or stabilise the monomeric form, and various fusion proteins that stabilise the monomeric form of IL-38. The present invention finds particular application reducing innate immune sensor-triggered type I IFN pathways, with affected mediators including (but not limited to) IRF3&7, IFNα, IFNβ, IFNλ, RANTES, CXCL10; the NF-kappa B pathway is also suppressed, albeit less efficiently. Type I IFNs play an important disease-augmenting role in a large number of diseases, but this role is critical in interferonopathies (IFNPs) such as systemic lupus, scleroderma, Sjögren syndrome, dermatomyositis and others.

As herein defined, an IL-38 polypeptide is a molecule that has at least one biochemical or biophysical activity of IL-38. For example, the polypeptide may be capable of binding to, and may be a ligand for IL-1R6 (also referred to as IL-36R or IL-1Rrp2). It may reduce IL-6 production in the context of inflammation. Other biochemical or biophysical activities of IL-38 may include blocking the production of pro-inflammatory cytokines (including IL-1β, IL-6, IL-8, IL-17A, IL-22, TNF, APRIL, CCL2 and others) triggered by a variety of inflammatory assaults. This activity may or may not be mediated by binding to IL-1R6. An IL-38 polypeptide may also bind to IL-1R10.

IL-38 is also thought to suppress Th 17 polarisation of T cells, including reduction of Th 17-associated cytokines such as IL-17A, IL-17F, IL-22, IL-23 and others. Anti-angiogenic effects and reduction of VEGF by IL-38 has been reported as well as an IL-38 mediated increase in PD-IL expression in cancer. A discussion of the proposed functions of IL-38 is reviewed in van de Veerdonk et al (2018) Immunol Rev, 281 (1): 191-196.

IL-38 is also known as interleukin-38 (or Interleukin-1 family member 10, ILIF10, Interleukin-1 theta, Interleukin-1 HY2, FIL1T, IL1HY2, FKSG75, UNQ6119/PRO20041). Any isoforms or orthologs of human IL-38 polypeptides which contain at least one residue equivalent to a dimer interface residue in SEQ ID NO: 1 are also contemplated within the present invention. Human IL-38 has 2 isoforms, which are alternative splice variants that result in a change in the amino acid sequence from residues 9 to 39 of SEQ ID NO: 1. Both isoforms are included when referring to IL-38 herein unless expressly stated otherwise. A further naturally occurring variant, contemplated within the scope of the present invention, is a polypeptide having the sequence of SEQ ID NO: 1, 2, 4 or 5 with a mutation at residue 44 from Isoleucine to Threonine (I44T using the numbering of SEQ ID NO: 1). Any isoforms or orthologs of human IL-38 polypeptides which contain at least one residue equivalent to a dimer interface residue as shown in Table 1 are also contemplated within the present invention. For example, the present invention includes polypeptides with identity to any of human IL-38 isoforms 1 and 2.

Further still, the present invention contemplates variations to the N-terminal and C-terminal region(s) of the polypeptides defined herein, for example in SEQ ID NO: 1, SEQ ID NO: 2, SEQ ID NO: 4 and SEQ ID NO: 5. Such N and/or C terminal variations may be included for the purposes of facilitating expression and/or purification of the polypeptides, and without impacting on the ability of the polypeptide to fold. The skilled person will be familiar with typical N- and C-terminal modifications, including the use of tags (including His-tags, FLAG tags and the like). Such tags may also be cleaved following purification of the polypeptide of the invention (for example, by include of a TEV protease cleavage sequence in the polypeptide). Similarly, an N-terminal methionine residue may be added for the purposes of facilitating recombinant expression (for example particularly in the case of any one SEQ ID NOs: 4 to 19).

As used herein, a dimerization interface refers to a region of a protein that enables dimerization of IL-38 monomers and refers to the interface between two IL-38 molecules which is involved in the stabilisation of the two interacting molecules. For example, the dimerization interface, in the context of IL-38, involves a domain-swap hinge region, which in the homodimer, is in the form of two alpha-helical regions (one in each monomer), enabling interaction of one molecule to the other. This region may be in the form of another motif (for example, a turn, including an Asx turn, a nest motif). The region corresponding to the 4-β5 loop (residues 43 to 58 of SEQ ID NO: 1) is a critical region of secondary structure that controls dimerization.

As used herein, an amino acid residue defined as being at a “position equivalent to” a position in a reference SEQ ID (eg SEQ ID NO: 1) can be determined by any means known to a person skilled in the art. For example, an alignment of one or more sequences with an amino acid sequence of the reference SEQ ID NO would allow a person skilled in the art to determine the amino acid at the position equivalent to position in the reference SEQ ID NO. For example, where the reference SEQ ID NO: is SEQ ID NO: 1, the skilled person can align the polypeptide in question with SEQ ID NO: 1, to determine what the equivalent residue is in the polypeptide in question. In more detail, given that In another example, SEQ ID NO: 4 is an N-terminally truncated variant of SEQ ID NO: 1. The skilled person would appreciate that the amino acid residue in SEQ ID NO: 4 that is at an equivalent position to residue 51 of SEQ ID NO: 1 (ie aspartate), is residue number 49 in SEQ ID NO: 4. a person skilled in the art can also compare the three dimensional structure of a polypeptide with the three dimensional structure of a polypeptide having the amino acid sequence of SEQ ID NO: 1 and determine the amino acid residue that is at an equivalent position to that in SEQ ID NO: 1.

As used herein “reduced capacity to form a dimer” refers to a lesser propensity for a polypeptide to form a homodimer and/or heterodimer compared to a reference polypeptide, when tested under the same conditions. The capacity for a polypeptide to form a dimer may be measured via various means including those methods described herein, such as size exclusion chromatography and multi-angle light scattering. Preferably, a polypeptide that has a reduced capacity to form a dimer has at least about a 10%, 20%, 30%, 40%, 50%, 60%, 70%, 80%, 90% or 100% reduction in dimer formation compared to a reference polypeptide. The reference polypeptide will typically be a native, wild-type, unmodified or unmutated polypeptide, for example a polypeptide comprising or consisting of the amino acid sequence of SEQ ID NO: 1. For the assessment of the proportion of a polypeptide present in a given form, i.e. a monomer or dimer, a suitable method is, for example, analytical gel electrophoresis under non-denaturing conditions. In such a method, a solution of the polypeptide is run in a polyacrylamide gel, alongside a set of standard molecular weight markers. If the polypeptide forms dimers, a protein band will be observed in the gel corresponding to a species with a molecular weight approximately twice that calculated for the sum of the amino acids of the polypeptide. A second band may also be observed corresponding to a species with approximately the molecular weight calculated for the sum of the amino acids of the polypeptide—this represents the sequence in monomeric form. The relative intensities of the bands may be used to quantify the proportion of the polypeptide which is present in each form. Similar methods may assess molecular weight by alternative means, for example, analytical centrifugation, mass spectrometry or size exclusion chromatography. Alternatively, monomers or dimers may be quantified using reverse phase high performance liquid chromatography (RP-HPLC) where the dimers and higher oligomeric species are separated from the monomers based on differences in their hydrophobicities. Identification of the species may be achieved using mass spectrometric detection. The same methods may be adapted to assess whether a given polypeptide shows a tendency to heterodimerise or homodimerise.

The anti-inflammatory properties of a polypeptide or fusion protein of the invention can be determined by any method described herein, particular in the Examples.

“Isolated,” when used to describe the various polypeptides disclosed herein, means the polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated protein includes polypeptide in situ within recombinant cells, since at least one component of the polypeptide natural environment will not be present. Ordinarily, however, isolated polypeptide will be prepared by at least one purification step.

A “fragment” is a portion of a polypeptide of the present invention that retains substantially similar functional activity or substantially the same biological function or activity as the polypeptide, which can be determined using assays described herein.

“Percent (%) amino acid sequence identity” or “percent (%) identical” with respect to a polypeptide sequence, i.e. a polypeptide of the invention defined herein, is defined as the percentage of amino acid residues in a candidate sequence that are identical with the amino acid residues in the specific polypeptide of the invention, after aligning the sequences and introducing gaps, if necessary, to achieve the maximum percent sequence identity, and not considering any conservative substitutions as part of the sequence identity.

Those skilled in the art can determine appropriate parameters for measuring alignment, including any algorithms (non-limiting examples described below) needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=X/Y100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of B to A.

In calculating percent identity, typically exact matches are counted. The determination of percent identity between two sequences can be accomplished using a mathematical algorithm. A non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. Sec Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, CA). After alignment of amino acid sequences with ClustalW, the percent amino acid identity can be assessed. A non-limiting examples of a software program useful for analysis of ClustalW alignments is GENEDOC™ or JalView (http://www.jalview.org/). GENEDOC™ allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, CA, USA). When utilizing the ALIGN program for comparing amino acid sequences, a PAM 120 weight residue table, a gap length penalty of 12, and a gap penalty of 4 can be used.

The polypeptide desirably comprises an amino end and a carboxyl end. The polypeptide can comprise D-amino acids, L-amino acids or a mixture of D- and L-amino acids. The D-form of the amino acids, however, is particularly preferred since a polypeptide comprised of D-amino acids is expected to have a greater retention of its biological activity in vivo.

The polypeptide can be prepared by any of a number of conventional techniques. The polypeptide can be isolated or purified from a naturally occurring source or from a recombinant source. Recombinant production is preferred. For instance, in the case of recombinant polypeptides, a DNA fragment encoding a desired peptide can be subcloned into an appropriate vector using well-known molecular genetic techniques (see, e.g., Maniatis et al., Molecular Cloning: A Laboratory Manual, 2nd ed. (Cold Spring Harbor Laboratory, 1982); Sambrook et al., Molecular Cloning A Laboratory Manual, 2nd ed. (Cold Spring Harbor Laboratory, 1989). The fragment can be transcribed and the polypeptide subsequently translated in vitro. Commercially available kits also can be employed (e.g., such as manufactured by Clontech, Palo Alto, Calif.; Amersham Pharmacia Biotech Inc., Piscataway, N.J.; In Vitrogen, Carlsbad, Calif., and the like). The polymerase chain reaction optionally can be employed in the manipulation of nucleic acids.

As used herein, the term “substitution” is intended to refer to replacement or change of the amino acid sequence of a peptide or polypeptide. It will be understood that occasionally the term “mutation” may also be used in the context of amino acid changes, and should be understood to be used interchangeably with the term “substitution” in the context of the present invention.

The term “conservative substitution” as used herein, refers to the replacement of an amino acid present in the native sequence in the peptide with a naturally or non-naturally occurring amino acid or a peptidomimetic having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

Conservative amino acid substitution tables providing functionally similar amino acids are well known to one of ordinary skill in the art. The following six groups are examples of amino acids that may be considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

As naturally occurring amino acids are typically grouped according to their properties, conservative substitutions by naturally occurring amino acids can be determined bearing in mind the fact that replacement of charged amino acids by sterically similar non-charged amino acids are considered as conservative substitutions. For producing conservative substitutions by non-naturally occurring amino acids it is also possible to use amino acid analogs (synthetic amino acids) well known in the art. A peptidomimetic of the naturally occurring amino acid is well documented in the literature known to the skilled person and non-natural or unnatural amino acids are described further below. When affecting conservative substitutions the substituting amino acid should have the same or a similar functional group in the side chain as the original amino acid.

The phrase “non-conservative substitution” or a “non-conservative residue” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, isoleucine for glycine, or —NH—CH[(—CH2)5-COOH]—CO— for aspartic acid. Non-conservative substitution includes any mutation that is not considered conservative.

A non-conservative amino acid substitution can result from changes in: (a) the structure of the amino acid backbone in the area of the substitution; (b) the charge or hydrophobicity of the amino acid; or (c) the bulk of an amino acid side chain. Substitutions generally expected to produce the greatest changes in protein properties are those in which: (a) a hydrophilic residue is substituted for (or by) a hydrophobic residue; (b) a proline is substituted for (or by) any other residue; (c) a residue having a bulky side chain, e.g., phenylalanine, is substituted for (or by) one not having a side chain, e.g., glycine; or (d) a residue having an electropositive side chain, e.g., lysyl, arginyl, or histadyl, is substituted for (or by) an electronegative residue, e.g., glutamyl or aspartyl.

Alterations of the native amino acid sequence to produce mutant polypeptides, such as by insertion, deletion and/or substitution, can be done by a variety of means known to those skilled in the art. For instance, site-specific mutations can be introduced by ligating into an expression vector a synthesized oligonucleotide comprising the modified site. Alternately, oligonucleotide-directed site-specific mutagenesis procedures can be used, such as disclosed in Walder et al., Gene 42:133 (1986); Bauer et al., Gene 37:73 (1985); Craik, Biotechniques, 12-19 (January 1995); and U.S. Pat. Nos. 4,518,584 and 4,737,462. A preferred means for introducing mutations is the QuikChange Site-Directed Mutagenesis Kit (Stratagene, LaJolla, Calif.).

Any appropriate expression vector (e.g., as described in Pouwels et al., Cloning Vectors: A Laboratory Manual (Elsevier, N.Y.: 1985)) and corresponding suitable host can be employed for production of recombinant polypeptides. Expression hosts include, but are not limited to, bacterial species within the genera Escherichia, Bacillus, Pseudomonas, Salmonella, mammalian or insect host cell systems including baculovirus systems (e.g., as described by Luckow et al., Bio/Technology 6:47 (1988)), and established cell lines such as the COS-7, C127, 3T3, CHO, HeLa, and BHK cell lines, and the like. The skilled person is aware that the choice of expression host has ramifications for the type of polypeptide produced. For instance, the glycosylation of polypeptides produced in yeast or mammalian cells (e.g., COS-7 cells) will differ from that of polypeptides produced in bacterial cells, such as Escherichia coli.

Alternately, a polypeptide of the invention can be synthesized using standard peptide synthesizing techniques well-known to those of ordinary skill in the art (e.g., as summarized in Bodanszky, Principles of Peptide Synthesis (Springer-Verlag, Heidelberg: 1984)). In particular, the polypeptide can be synthesized using the procedure of solid-phase synthesis (see, e.g., Merrifield, J. Am. Chem. Soc. 85:2149-54 (1963); Barany et al., Int. J. Peptide Protein Res. 30:705-739 (1987); and U.S. Pat. No. 5,424,398). If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The polypeptide-containing mixture can then be extracted, for instance, with dimethyl ether, to remove non-peptidic organic compounds, and the synthesized polypeptide can be extracted from the resin powder (e.g., with about 25% w/v acetic acid). Following the synthesis of the polypeptide, further purification (e.g., using high performance liquid chromatography (HPLC)) optionally can be done in order to eliminate any incomplete polypeptides or free amino acids. Amino acid and/or HPLC analysis can be performed on the synthesized polypeptide to validate its identity. For other applications according to the invention, it may be preferable to produce the polypeptide as part of a larger fusion protein, such as by the methods described herein or other genetic means, or as part of a larger conjugate, such as through physical or chemical conjugation, as known to those of ordinary skill in the art and described herein.

A polypeptide of the invention may also be modified by, conjugated or fused to another moiety to facilitate purification, or increasing the in vivo half-life of the polypeptides, or for use in immunoassays using methods known in the art. For example, a polypeptide of the invention may be modified by glycosylation, acetylation, pegylation, phosphorylation, amidation, derivatization by known protecting/blocking groups, proteolytic cleavage, linkage to a cellular ligand or other protein, etc.

A “peptidomimetic” is a synthetic chemical compound that has substantially the same structure and/or functional characteristics of a polypeptide of the invention, the latter being described further herein. Typically, a peptidomimetic has the same or similar structure as a polypeptide of the invention, for example the same or similar sequence of SEQ ID NO: 1 or fragment thereof that has a reduced capacity to form a dimer. A peptidomimetic generally contains at least one residue that is not naturally synthesised. Non-natural components of peptidomimetic compounds may be according to one or more of: a) residue linkage groups other than the natural amide bond (‘peptide bond’) linkages; b) non-natural residues in place of naturally occurring amino acid residues; or c) residues which induce secondary structural mimicry, i.e, to induce or stabilize a secondary structure, e.g., a beta turn, gamma turn, beta sheet, alpha helix conformation, and the like.

Peptidomimetics can be synthesized using a variety of procedures and methodologies described in the scientific and patent literatures, e.g., Organic Syntheses Collective Volumes, Gilman et al. (Eds) John Wiley & Sons, Inc., NY, al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymot. 267:220-234

Modifications contemplated herein include, but are not limited to, modification to side chains, incorporating of unnatural amino acids and/or their derivatives during polypeptide synthesis and the use of crosslinkers and other methods which impose conformational constraints on the polypeptides of the invention. Any modification, including post-translational modification that reduces the capacity of the molecule to form a dimer is contemplated herein. An example includes modification incorporated by click chemistry as known in the art. Exemplary modifications include PEGylation and glycosylation.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourca formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 2.

TABLE 2

| Non-conventional | |
|---|---|
| amino acid | Code |
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| y-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl- | Nmbc |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-y-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyl-α-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Crosslinkers can be used, for example, to stabilise 3D conformations, using homo-bifunctional crosslinkers such as the bifunctional imido esters having (CH2) n spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

### Fusion Proteins

The aforementioned IL-38 polypeptides are preferably provided in the form of a fusion protein, ie, wherein the IL-38 polypeptide is linked or joined to an Fc region of an antibody.

The term “antibody” refers to various antibody structures, including but not limited to monoclonal antibodies, polyclonal antibodies, multispecific antibodies. The term “antibody” may also be used interchangeably with the term “immunoglobulin”.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Particularly, the term “recombinant human antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Thus, such recombinant human antibodies have variable and constant regions derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “Fc region” herein is used to define a C-terminal region of an immunoglobulin heavy chain that contains at least a portion of the constant region. In other words, the Fc region contains two heavy chain fragments comprising the C_{H}2 and C_{H}3 domains of an antibody. In the context of the present invention, the Fc region comprises two heavy chain fragments, preferably the CH2 and CH3 domains of said heavy chain. The two heavy chain fragments are held together by two or more disulfide bonds and by hydrophobic interactions of the CH3 domains. The heavy chain constant domains that correspond to the different classes of immunoglobulins are called «, δ, ε, γ, and μ, respectively.

The genetic fusion of proteins, such as ligand-binding soluble receptors, cytokines, growth factors, enzymes, and peptides, to the Fc domain of human IgG1 is a known and about 11 Fc-fusion proteins have been clinically approved. All of the marketed Fc-fusion proteins and many others in clinical trials are based on the wild-type human IgG1 Fc, which presents the fusion partner as a homodimer due to the homodimeric Fc structure. However, in the context of certain preferred embodiments of present invention, and given the findings of the inventors in relation to the activity of monomeric IL-38, it may be desirable to retain a monomeric, or substantially monomeric configuration of the fusion partner (ie IL-38 polypeptide). To achieve this, the invention proposes the use of a heterodimeric Fc as an alternative scaffold to wild-type Fc.

Accordingly, in preferred examples, the fusion proteins of the invention comprise Fc regions which are not capable of homodimerisation. Suitable technology for preventing homodimerisation of Fc regions of antibodies is well known and in some examples may include the use of “knob-into-hole” technology which enables the heterodimerisation of the Fc region of the fusion protein such that the “paired” Fc region does not comprise an IL-38 polypeptide or may comprise an alternative polypeptide fused thereto. Examples of suitable “knob-into-hole” Fc sequences for use in generating monomeric fusion proteins are described in WO 2012/106587 (US20140079689), and also in Ridgeway et al., (1996) Protein Engineering, 9:617-621; incorporated herein by reference.

In some aspects, the fusion protein of the invention does not exhibit any effector function or any detectable effector function. “Effector functions” or “effector activities” refer to those biological activities attributable to the Fc region of an antibody, which vary with the antibody isotype. Examples of antibody effector functions include: C1q binding and complement dependent cytotoxicity (CDC); Fc receptor binding; antibodydependent cell-mediated cytotoxicity (ADCC); phagocytosis; down regulation of cell surface receptors (e.g. B cell receptor); and B cell activation. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinect, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennic, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831). Further, alterations may be made in the Fc region that result in altered (i.e., diminished) C1q binding and/or Complement Dependent Cytotoxicity (CDC), e.g., as described in U.S. Pat. No. 6,194,551, WO 99/51642, and Idusogic et al. J. Immunol. 164:4178-4184 (2000).

In some aspects, the Fc region includes mutations to the complement (Clq) and/or to Fc gamma receptor (FcγR) binding sites. In some aspects, such mutations can render the fusion protein incapable of antibody directed cytotoxicity (ADCC) and complement directed cytotoxicity (CDC).

The Fc region as used in the context of the present invention does not trigger cytotoxicity such as antibody-dependent cellular cytotoxicity (ADCC) or complement dependent cytotoxicity (CDC).

The term “Fc region” also includes native sequence Fc regions and variant Fc regions. The Fc region may include the carboxyl-terminus of the heavy chain. Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. Amino acid sequence variants of the Fc region of an antibody may be contemplated. Amino acid sequence variants of an Fc region of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the Fc region of the antibody. Any combination of deletion, insertion, and substitution can be made to arrive at the final construct, provided that the final construct possesses the desired characteristics, e.g., inducing or supporting an anti-inflammatory response.

The Fc region of the antibody may be an Fc region of any of the classes of antibody, such as IgA, IgD, IgE, IgG, and IgM. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA₁, and IgA₂. Accordingly, as used in the context of the present invention, the antibody may be an Fc region of an IgG. For example, the Fc region of the antibody may be an Fc region of an IgG1, an IgG2, an IgG2b, an IgG3 or an IgG4. In some aspects, the fusion protein of the present invention comprises an IgG of an Fc region of an antibody. In the context of the present invention, the Fc region of the antibody is an Fc region of an IgG, preferably IgG1.

Moreover, the herein provided fusion proteins may comprise a linker (or “spacer”). In the context of the present invention, the polypeptide (i.e. IL-38 polypeptide) is fused via a linker at the C-terminus to the Fc region. A linker is usually a peptide having a length of up to 20 amino acids. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 amino acids. For example, the herein provided fusion protein may comprise a linker between the IL-38 polypeptide and the Fc region of the antibody, such as between the N-terminus of the Fc regions and the C-terminus of the IL-38 polypeptide. As another example, the herein provided fusion protein may comprise a linker between the IL-37 polypeptide and the Fc region of the antibody, such as between the C-terminus of the Fc regions and the N-terminus of the IL-38 polypeptide. Particularly, the IL-38 polypeptide may be fused via a linker at the C-terminus to the N-terminus of the Fc region. Such linkers have the advantage that they can make it more likely that the different polypeptides of the fusion protein fold independently and behave as expected. Thus, in the context of the present invention the IL-38 polypeptide and the Fc region of an antibody may be comprised in a single-chain multi-functional polypeptide. In some aspects, the fusion protein of the present invention includes a peptide linker. In some aspects, the peptide linker links an IL-38 polypeptide with an Fc region of an antibody. In some aspects, the peptide linker can include the amino acid sequence Gly-Gly-Ser (GGS), Gly-Gly-Gly-Ser (GGGS) or Gly-Gly-Gly-Gly-Ser (GGGGS). In some aspects, the peptide linker can include the amino acid sequence GGGGS (a linker of 6 amino acids in length) or even longer. The linker may a series of repeating glycine and serine residues (GS) of different lengths, i.e., (GS), where n is any number from 1 to 15 or more. For example, the linker may be (GS) 3 (i.e., GSGSGS) or longer (GS) n or longer. It will be appreciated that n can be any number including 1, 2, 3, 4, 5, 6, 7, 8, 9, 10, 11 or more. Fusion proteins having linkers of such length are included within the scope of the present invention. Preferably n is no more than 3 (ie such that when n equals 3 the linker is GSGSGS).

Other exemplary linkers include proline rich linkers such as the sequence PAPAP, APAPA and variations thereof.

In certain embodiments, the IL-38 polypeptide is directly fused to the Fc region of an antibody, such that there is no linker between the two regions of the fusion protein.

Nucleic acid molecules that encode any of the polypeptides of the invention are also within the scope of the invention. The nucleic acids are useful, for example, in making the polypeptides of the present invention and as therapeutic agents. They may be administered to cells in culture or in vivo and may include a secretory signal that directs or facilitates secretion of the polypeptide of the invention from the cell. Also within the scope of the invention are expression vectors and host cells that contain or include nucleic acids of the invention (described further below). While the nucleic acids of the invention may be referred to as “isolated,” by definition, the polypeptides of the invention are not wild-type polypeptides and, as such, would not be encoded by naturally occurring nucleic acids. Thus, while the polypeptides and nucleic acids of the present invention may be “purified,” “substantially purified,” “isolated,” “recombinant” or “synthetic” they need not be so in order to be distinguished from naturally occurring materials.

An “isolated” nucleic acid molecule is a nucleic acid molecule that is identified and separated from at least one contaminant nucleic acid molecule with which it is ordinarily associated in the natural source of the polypeptide encoding, typically IL-38, nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes nucleic acid molecules contained in cells that ordinarily express IL-38 where, for example, the nucleic acid molecule is in a chromosomal location different from that of natural cells.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or purified form. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides of the invention can be synthesised according to methods well known in the art, as described by way of example in Sambrook et al (1989, Molecular Cloning—a laboratory manual; Cold Spring Harbor Press).

The polynucleotide molecules of the present invention may be provided in the form of an expression cassette which includes control sequences operably linked to the inserted sequence, thus allowing for expression of the polypeptide of the invention in vivo in a targeted subject. These expression cassettes, in turn, are typically provided within vectors (e.g., plasmids or recombinant viral vectors) which are suitable for use as reagents for nucleic acid immunization. Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Thus, the present invention provides a vector for use in preventing or treating an inflammatory disease or condition comprising a polynucleotide sequence which encodes a polypeptide of the invention and optionally one or more further polynucleotide sequences which encode different polypeptides as defined herein.

Furthermore, it will be appreciated that the compositions and products of the invention may comprise a mixture of polypeptides and polynucleotides. Accordingly, the invention provides a composition or product as defined herein, wherein in place of any one of the polypeptide is a polynucleotide capable of expressing said polypeptide.

Expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al.

Thus, a polypeptide of the invention may be provided by delivering such a vector to a cell and allowing transcription from the vector to occur. Preferably, a polynucleotide of the invention or for use in the invention in a vector is operably linked to a control sequence which is capable of providing for the expression of the coding sequence by the host cell, i.e. the vector is an expression vector.

“Operably linked” refers to an arrangement of elements wherein the components so described are configured so as to perform their usual function. Thus, a given regulatory sequence, such as a promoter, operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

A number of expression systems have been described in the art, each of which typically consists of a vector containing a gene or nucleotide sequence of interest operably linked to expression control sequences. These control sequences include transcriptional promoter sequences and transcriptional start and termination sequences. The vectors of the invention may be for example, plasmid, virus or phage vectors provided with an origin of replication, optionally a promoter for the expression of the said polynucleotide and optionally a regulator of the promoter. A “plasmid” is a vector in the form of an extra-chromosomal genetic element. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polypeptide.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

A polynucleotide, expression cassette or vector according to the present invention may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter.

Typically a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allowing for secretion of the polypeptide via the Golgi apparatus. Thus, a peptide of the invention may be provided to an individual by expression from cells within the individual, and secretion from those cells.

The phrase “therapeutically effective amount” generally refers to an amount of one or more polypeptides or polynucleotides of the invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein.

The invention finds application in the treatment of various inflammatory diseases or conditions for which treatment with recombinant wild-type IL-38 has been suggested, for example, for the treatment of systemic lupus erythematosus (SLE), scleroderma, Sjogren's syndrome, polymyositis, dermatomyositis, autoimmune haemolytic anaemia, immune thrombocytopaenia, neuromyelitis optica, myasthenia gravis, alopecia areata, cutaneous lupus, discoid lupus, inherited interferonopathies, male or female infertility, rheumatoid arthritis (RA), psoriatic arthritis (PsA), ankylosing spondylitis, psoriasis, Addison's disease, autoimmune hepatitis, autoimmune parotitis, Crohn's disease, diabetes mellitus (Type I), dystrophic epidermolysis bullosa, epididymitis, glomerulonephritis, Graves' disease, Guillian-Barre syndrome, Hashimoto's disease, multiple sclerosis, pemphigus vulgaris, rheumatic fever, sarcoidosis, spondyloarthropathies, thyroiditis, vasculitis, vitiligo, myxedema, pernicious anemia, ulcerative colitis, and related conditions.

The present invention also finds application in the treatment of interferonopathies; a group of autoinflammatory disorders associated with prominent enhanced type I interferon signalling. Examples of such disorders include Aicardi-Goutières syndrome, spondyloenchondro-dysplasia with immune dysregulation, stimulator of interferon genes-associated vasculopathy with onset in infancy, X-linked reticulate pigmentary disorder, ubiquitin-specific peptidase 18 deficiency, chronic atypical neutrophilic dermatitis with lipodystrophy, and Singleton-Merten syndrome. Such conditions or syndromes may be characterised by intracranial calcification, skin vasculopathy, interstitial lung disease, failure to thrive, skeletal development problems, autoimmune features, abnormal responses to nucleic acid stimuli and defective regulation of protein degradation.

For any of the diseases or conditions described herein, when the polypeptide of the present invention is topically administered to a human, the therapeutically effective amount of a compound corresponds to preferably between about 0.01 to about 10% (w/w), or between about 0.1 to 10% (w/w), or between about 1.0 to about 10% (w/w), between about 0.1 to about 5% (w/w), or between about 1.0 to about 5% (w/w). In any of the diseases or conditions diseases described herein, when the polypeptide of the present invention is orally administered to a subject, the therapeutically effective amount of a compound corresponds preferably between about 1 to about 50 mg/kg, or between about 1 to 25 mg/kg, or between about 1 to about 10 mg/kg, between about 5 to about 25 mg/kg, or between about 10 to about 20 mg/kg.

In a composition of the invention, the proportion of polypeptide of the invention present as a monomer may be at least about 0.5%, 1%, 2%, 3%, 4%, 5%, 6%, 7%, 8%, 9%, 10%, 20%, 30%, 40%, 50%, 60%, 70%, 80% or 90% of the total polypeptide present in the composition, typically stored in solution for a suitable period of time under suitable conditions. Suitable periods of time and conditions include ranges of time and conditions under which a skilled practitioner might reasonably expect to keep a polypeptide in solution prior to use. For example, periods of time of about 24 hours, about 48 hours, or about 72 hours are typical, although some solutions may be kept for longer periods for example, at least a week, a month, 6 months, 1 year, 2 years, 3 years or more. Storage conditions may typically be room temperature and relative humidity, or typically 25° C. and 60% relative humidity, but could include any standard storage conditions encountered by the skilled person, for example approximately 4° C., −20° C., or −80° C.

The frequency of administration may be once daily, or 2 or 3 time daily. The treatment period may be for the duration of the detectable disease.

Typically, a therapeutically effective dosage is formulated to contain a concentration (by weight) of at least about 0.1% up to about 50% or more, and all combinations and sub-combinations of ranges therein. The compositions can be formulated to contain one or more polypeptides of the invention in a concentration of from about 0.1 to less than about 50%, for example, about 49, 48, 47, 46, 45, 44, 43, 42, 41 or 40%, with concentrations of from greater than about 0.1%, for example, about 0.2, 0.3, 0.4 or 0.5%, to less than about 40%, for example, about 39, 38, 37, 36, 35, 34, 33, 32, 31 or 30%. Exemplary compositions may contain from about 0.5% to less than about 30%, for example, about 29, 28, 27, 26, 25, 25, 24, 23, 22, 21 or 20%, with concentrations of from greater than about 0.5%, for example, about 0.6, 0.7, 0.8, 0.9 or 1%, to less than about 20%, for example, about 19, 18, 17, 16, 15, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 5%. In all cases, amounts may be adjusted to compensate for differences in amounts of active ingredients actually delivered to the treated cells or tissue.

Although the invention finds application in humans, the invention is also useful for therapeutic veterinary purposes. The invention is useful for domestic or farm animals such as cattle, sheep, horses and poultry; for companion animals such as cats and dogs; and for zoo animals.

Further, the present invention may find particular application in the treatment of individuals who are at greater risk of developing a disease or condition associated with reduced endogenous IL-38 function. For example, while it is intended that the polypeptides, pharmaceutical compositions and methods of the present invention, may be used for treating inflammatory conditions in any individual in need thereof, the present invention also contemplates a particular use in treating inflammatory conditions in a subject who is homozygous or heterozygous for an IL-38 allele that encodes a form of the protein that has a greater propensity to form a dimer than a polypeptide of SEQ ID NO: 2. More specifically, the SNP rs6743376 is a missense variant (C to A single nucleotide variation) that results in a change in the nucleic acid sequence of the gene encoding IL-38 in humans. The result of the missense variation is a change in the amino acid sequence at residue 51 from Alanine to Aspartic Acid. According to the findings of the present inventors, individuals homozygous for the “wild-type” sequence (C:C) or heterozygous (C; A) are anticipated to produce forms of IL-38 that have decreased monomer stability and an increased propensity to form dimers and are anticipated to be at greater risk of developing inflammatory disease. Indeed, population studies have already identified that individuals carrying the SNP are at a decreased risk of arthritis. Accordingly, although not so limited, the methods of the present invention may find particular application in treating inflammatory conditions in individuals who are homozygous or heterozygous “wild-type” (i.e., bearing the C; C or C; A allele at rs6743376).

Accordingly, in still further embodiments, the methods of the present invention may include determining whether a subject may be susceptible to requiring treatment with a polypeptide or composition of the invention, wherein the step of determining may include assessing the genotype of the individual at SNP rs6743376. These methods may include obtaining a sample of genomic DNA from the subject and sequencing across the SNP locus; or alternatively, observing such information in a clinical report provided by a 3^{rd} party who has determined the genotype of the individual. Such genotyping and sequencing methods will be well known to the person skilled in the present art.

Pharmaceutical compositions may be formulated for any appropriate route of administration including, for example, topical (for example, transdermal or ocular), oral, buccal, nasal, vaginal/intrauterine, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. Within yet other embodiments, compositions provided herein may be formulated as a lyophilizate.

The various dosage units are each preferably provided as a discrete dosage tablet, capsules, lozenge, dragée, gum, or other type of solid formulation. Capsules may encapsulate a powder, liquid, or gel. The solid formulation may be swallowed, or may be of a suckable or chewable type (either frangible or gum-like). The present invention contemplates dosage unit retaining devices other than blister packs; for example, packages such as bottles, tubes, canisters, packets. The dosage units may further include conventional excipients well-known in pharmaceutical formulation practice, such as binding agents, gellants, fillers, tableting lubricants, disintegrants, surfactants, and colorants; and for suckable or chewable formulations.

Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatine or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatine capsules wherein the active ingredient is mixed with an inert solid diluent such as calcium carbonate, calcium phosphate or kaolin, or as soft gelatine capsules wherein the active ingredient is mixed with water or an oil medium such as peanut oil, liquid paraffin or olive oil.

Aqueous suspensions contain the active ingredient(s) in admixture with excipients suitable for the manufacture of aqueous suspensions. Such excipients include suspending agents such as sodium carboxymethylcellulose, methylcellulose, hydropropylmethylcellulose, sodium alginate, polyvinylpyrrolidone, gum tragacanth and gum acacia, and dispersing or wetting agents such as naturally-occurring phosphatides (for example, lecithin), condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol mono-oleate, or condensation products of ethylene oxide with partial esters derived from fatty acids and hexitol anhydrides such as polyethylene sorbitan monooleate. Aqueous suspensions may also comprise one or more preservatives, for example ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil such as arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavouring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavouring and colouring agents, may also be present.

Pharmaceutical compositions may also be in the form of oil-in-water emulsions. The oily phase may be a vegetable oil such as olive oil or arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavouring agents.

Syrups and elixirs may be formulated with sweetening agents, such as glycerol, propylene glycol, sorbitol or sucrose. Such formulations may also comprise one or more demulcents, preservatives, flavouring agents and/or colouring agents.

Polypeptides of the invention may be formulated for local or topical administration, such as for topical application to the skin. Formulations for topical administration typically comprise a topical vehicle combined with active agent(s), with or without additional optional components.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturizers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminum silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylceilulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions may be formulated for transdermal administration (for example, as a transdermal patch). Topical compositions may also be in the form of coatings for implantable devices including catheters, venous access devices or surgical meshes. Other topical compositions contemplated by the present invention include compositions formulated for ophthalmic use, including formulation as eye drops.

A pharmaceutical composition may be formulated as inhaled formulations, including sprays, mists, or aerosols. For inhalation formulations, the composition or combination provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient.

Pharmaceutical compositions may also be prepared in the form of suppositories such as for vaginal or rectal administration. Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions may be formulated as sustained release formulations such as a capsule that creates a slow release of modulator following administration. Such formulations may generally be prepared using well-known technology and administered by, for example, oral, rectal or subcutaneous implantation, or by implantation at the desired target site. Carriers for use within such formulations are biocompatible, and may also be biodegradable. Preferably, the formulation provides a relatively constant level of modulator release. The amount of modulator contained within a sustained release formulation depends upon, for example, the site of implantation, the rate and expected duration of release and the nature of the condition to be treated or prevented.

In another embodiment, there is provided a kit or article of manufacture including one or more polypeptides or polynucleotides of the invention and/or pharmaceutical composition as described above.

In other embodiments, there is provided a kit for use in a therapeutic or prophylactic application mentioned above, the kit including:
a container holding a polypeptide, polynucleotide or pharmaceutical composition of the invention;
a label or package insert with instructions for use.

In certain embodiments, the kit may contain one or more further active principles or ingredients for treatment of inflammatory diseases or conditions.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic or prophylactic composition can be used to treat an inflammatory disease or condition described herein.

The kit may comprise (a) a therapeutic or prophylactic composition; and (b) a second container with a second active principle or ingredient contained therein. The kit in this embodiment of the invention may further comprise a package insert indicating the composition and other active principle can be used to treat a disorder or prevent a complication stemming from an inflammatory disease or condition described herein. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain embodiments the therapeutic composition may be provided in the form of a device, disposable or reusable, including a receptacle for holding the therapeutic, prophylactic or pharmaceutical composition. In one embodiment, the device is a syringe. The device may hold 1-2 mL of the therapeutic composition. The therapeutic or prophylactic composition may be provided in the device in a state that is ready for use or in a state requiring mixing or addition of further components.

A polypeptide or composition of the invention may be used as a coating for implantable materials and devices, e.g. stents. The polypeptide or composition of the invention may be coated on to, or integral with, the implantable material or device. The polypeptide or composition of the invention may be part of a polymeric coating.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1: IL-38 Cloning, Bacterial Expression, and Purification for Crystallography and Biophysical Characterisation

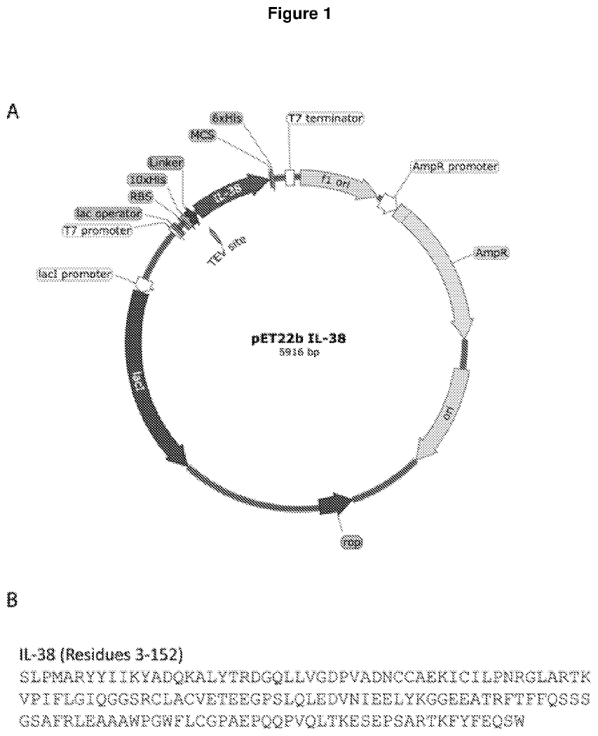

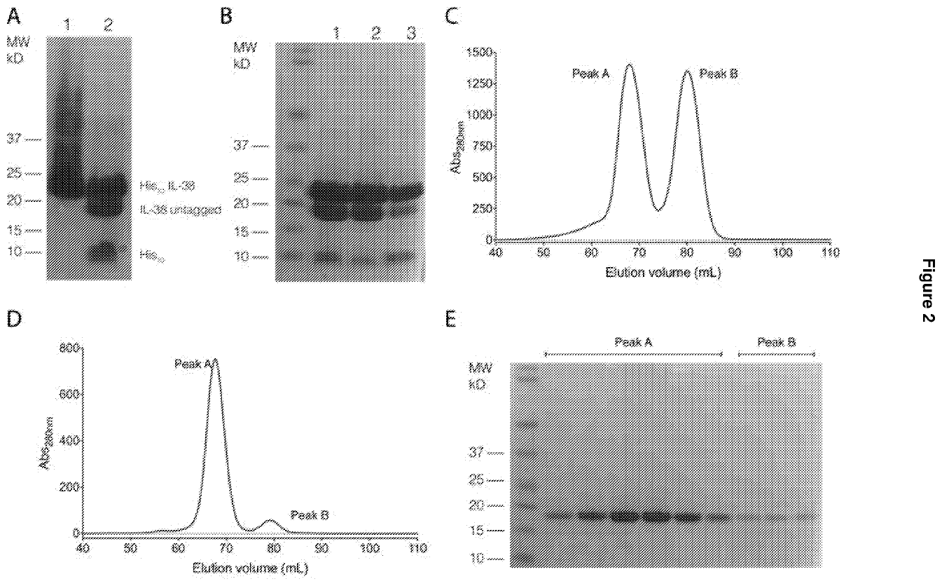

Codon optimised IL-38 (residues 3-152) was synthesised and cloned (Genscript) into pET22b(+) (Novagen). Vector map of pET22b(+) IL-38 shown in FIG. 1A and amino acid sequence of residues 3-152 of WT IL-38 (SEQ ID NO: 4). A His₁₀-tagged sequence followed by a tobacco etch virus (TEV) cleavable sequence was added at the N-terminus of the IL-38 sequence to facilitate purification.

Recombinant IL-38 comprising the sequence of SEQ ID NO: I was expressed in BL21-CodonPlus(DE3)-RIL cells (Stratagene) by IPTG induction (OD₆₀₀ 0.8) overnight at 18° C. The cells were lysed by sonication in 50 mM Tris-HCl (pH 8.0), 500 mM NaCl, 3 mM β-mercaptoethanol, 25 mM imidazole, with two complete EDTA-free protease inhibitor tablets (Roche). Cells were clarified by centrifugation, filtered through a 0.45 μm membrane, and the soluble fraction bound to Ni-NTA resin (Qiagen) for 1 hour at 4° C. The resin was washed with 500 ml of 50 mM Tris-HCl (pH 8.0), 250 mM NaCl, 3 mM B-mercaptoethanol, and 25 mM imidazole. After nickel elution, IL-38 was highly pure and subsequent overnight TEV cleavage resulted in removal of the His₁₀ tag from approximately 50% of the protein (FIG. 2A).

The eluted protein was loaded onto a 1 ml HiTrapQ column and eluted with a NaCl gradient from 0.05 M to 1 M NaCl. The eluted protein was analysed by SDS-PAGE and contained both cleaved and uncleaved IL-38 (FIG. 2B). IL-38 was loaded onto a Superdex 75 16/60 column (Cytiva Life Sciences) and eluted in two peaks with an elution volume indicated a size corresponding to monomeric and dimeric IL-38 conformations (FIG. 2C). Uncut IL-38 was removed from the Peak A protein pool by further incubation with Ni-NTA resin and the untagged IL-38 was further purified by SEC (FIG. 2D). SDS-PAGE analysis of SEC run (FIG. 2E).

### Example 2: Analytical SEC Analysis of IL-38, Mechanism of Dimer Formation

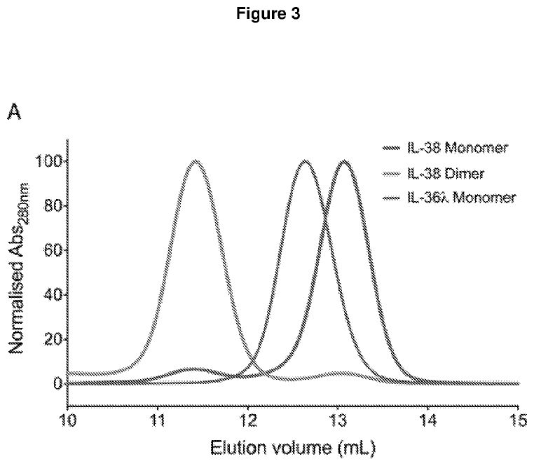

Purified IL-38 from Peak A (orange) and Peak B (blue) was analysed on an analytical Superdex 75 10/300 GL column (Cytiva Life Sciences) to give an indication of the size of the two protein conformations (FIG. 3). Monomeric recombinant IL-36λ was loaded onto the column as a control. The IL-38 Peak A eluted off the column at a volume indicative of a homodimer and IL-38 Peak B eluted off the column at a volume indicative of a monomer. Importantly, neither Peak A or Peak B of IL-38 readily interconverted during storage or SEC. These data indicate that Peak A and Peak B are not in rapid equilibrium. A traditional head-to-head homodimer (e.g. IL-37) would be expected to dissociate back into a monomer, especially at concentrations whereby both monomer and dimer are observed during purification.

SEC characterisation of IL-38 suggests a domain-swapping mechanism of homodimer formation. There are three major types of protein dimerization are regularly observed in vitro and in vivo. They include head-to-head, head-to-tail, and domain-swapping. In head-to-head homodimers, the same region of each protein interacts to form a symmetrical and closed conformation. In head-to-tail dimerization different regions of the proteins interact forming an open conformation that leads to oligomerisation. As such, a stable dimeric conformation is not regularly observed in head-to-tail oligomerization. Lastly, domain-swapping occurs when regions/domains of each protein subunit swap positions to form a highly stable homodimer.

FIG. 2 (C-D) shows that IL-38 displays the typical behaviour of a domain-swapped dimer during SEC. Firstly, upon SEC IL-38 partitions into two peaks that correspond to a homodimer and a monomer. Initially, this would suggest that IL-38 is at a concentration near the dimerization constant whereby a rapid and even equilibrium is observed between monomeric and homodimeric conformations. However, crucially upon further purification and dilution the IL-38 homodimer does not readily dissociate back into a monomeric population (D). This is typical behaviour of a domain-swapped dimer and is indicative of two IL-38 populations (monomer and domain-swapped dimer) that do not readily interconvert.

### Size Exclusion Chromatography—Multiangle Light Scattering (SEC-MALS)

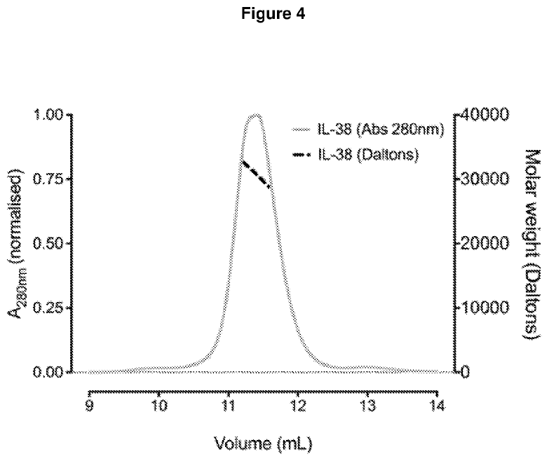

SEC-MALS measurements were carried out on a Superdex 75 10/300 column (Cytiva Life Sciences) equilibrated in 10 mM Hepes (pH 7.3), 100 mM NaCl, 1 mM EDTA, and 2 mM DTT (FIG. 4). All experiments were conducted at 25° C. at a flow rate of 0.4 ml/min in the above buffer. A volume of 110 μl of IL-38 (WT) at 6 mg/ml was injected for the run. Test injections of bovine serum albumin (2 mg/ml; Thermo Scientific Pierce) were used for calibration purposes. The SEC-MALS system is composed of a Shimadzu DGU-20A5 degasser, an LC-20AD liquid chromatograph, a SIL-20AHT autosampler, a CBM-20A communications bus module, an SPD-20A UV/VIS detector, and a CTO-20AC column oven, which was coupled to a DAWN HELEO-II MALS detector fitted with an Optilab T-rEX refractive index detector (Wyatt Technology). Molar mass was calculated by measuring the intensity of scattered light at 18 different scattering angles. Molecular mass calculations were performed using Astra 6.1 software (Wyatt Technology).

### Example 3: Crystallisation of IL-38 Dimer

IL-38 crystals were grown at 20° C. by hanging drop vapour diffusion in 1.2 M NaH₂PO₄, 0.8 M K₂HPO₄, 0.2 M LiSO₄, and 0.1 M N-cyclohexyl-3-aminopropanesulfonic acid (pH 10.5) at a 1:1 drop ratio. Crystals were flash cooled in liquid nitrogen in mother liquor containing 30% (v/v) glycerol. X-ray data were collected at the MX2 beamline (microfocus) of the Australian Synchrotron at a wavelength of 0.95373. Data were processed and scaled using XDS and programs within the CCP4 suite. The structure was solved by molecular replacement using monomeric IL-38 (PDB code 5BOW) as the search model in PHENIX using the Phaser program. The initial structure was rebuilt manually in COOT and iterative cycles of refinement were carried out using twinned refinement in REFMAC5 with local rebuilding in COOT, resulting in a model with an R-factor of 21.29% (R_{free} of 27.44%) and good geometry (Table 3). The structure had no Ramachandran outliers with 91.05% of residues in favored regions.

TABLE 3
Data collection and refinement statistics

| | IL-38 (WT) | IL-38 (ΔT53) |
|---|---|---|
| Data collection | | |
| Space group | P32 | P31 |
| Cell dimensions | | |
| a, b, c (Å) | 59.63, 59.79, 95.84 | 57.82, 57.82, 75.34 |
| α, β, γ (°) | 90.01, 90.04, | 90, 90, 120 |
| | 119.83 | |
| Resolution (Å) | 31.95-3.40 (3.67- | 20.08-1.7 (1.73- |
| | 3.40)* | 1.7)* |
| R_{merge} | 9.1 (177.9) | 11.2 (163.3) |
| I/σI | 15.3 (1.4) | 13.9 (2.7) |
| Completeness (%) | 99.8 (99.6) | 100 (100) |
| Redundancy | 10.6 (10.0) | 10.6 (8.8) |
| Refinement | | |
| Resolution (Å) | 29.88-3.40 | 30.22-1.75 |
| No. reflections | 4751 (359) | 28675 (2867) |
| R_{work}/R_{free} | 21.29 (27.44) | 16.64 (19.13) |
| No. atoms | | |
| Protein | 1657 | 2308 |
| Ligand/ion | — | 26 |
| Water | — | 59 |
| B-factors | | |
| Protein | 140.497 | 36.15 |
| Ligand/ion | — | 60.02 |
| Water | — | 39.83 |
| r.m.s. deviations | | |
| Bond lengths (Å) | 0.005 | 0.018 |
| Bond angles (°) | 1.335 | 1.52 |
| Ramachandran: favored, | 91.05, 8.95, 0 | 97.24, 2.76, 0 |
| allowed, outliers (%) | 21.08 | 1.73 |
| Clash score | | |

*Values in parentheses are for highest-resolution shell and each dataset is from a single crystal.

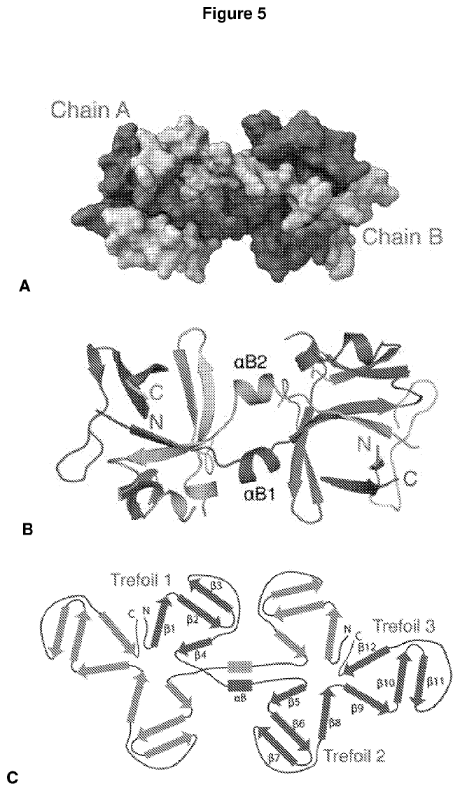

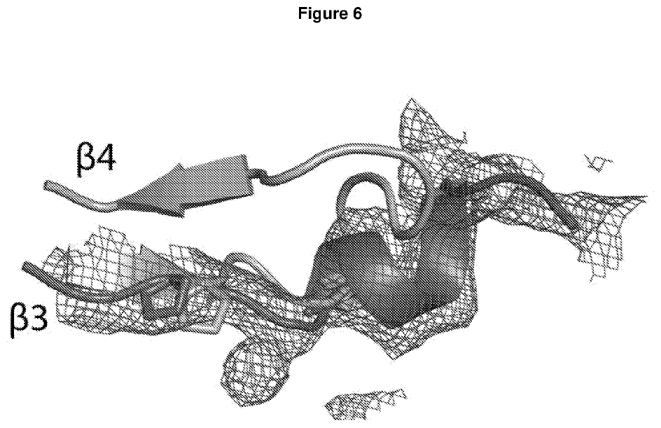

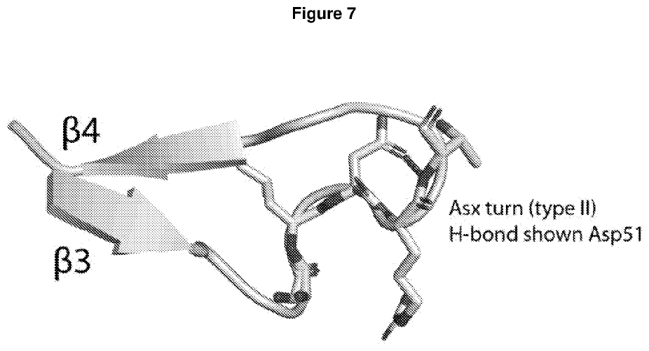

FIGS. 5A, B and C shows that solved structure of the IL-38 homodimer. FIG. 6 shows a secondary structure alignment of the IL-38 monomer with the IL-38 dimer, highlighting the hinge region which forms the dimerization interface (domain-swap hinge). In contrast to the monomer, the domain swap region in the dimer is characterised by the formation of an alpha helix. FIG. 7 shows that in the monomer, a loop is formed between the β4 and β5 strands.

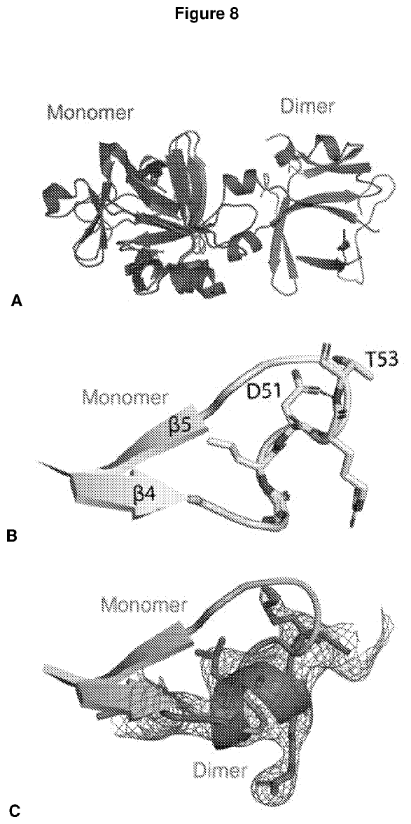

Comparison of the IL-38 monomeric and dimeric structures reveals the mechanism of dimer formation (FIG. 8A). The IL-38 monomer structure is of a SNP (rs6743376) with a mutation at residue 51 (A51D). (FIG. 8B) & (FIG. 8C) The solved IL-38 homodimer structure is the WT (D51A) sequence with an alanine at position 51. Aspartic acid at position 51 stabilises the β-turn reducing dimer formation.

### Example 4: Expression, Purification and Structural Characterisation of IL-38 Polypeptides Variants

### Purification and SEC Analysis

Codon-optimized IL-38 variants (residues 3-152, with the addition of an initiator methionine) were synthesised and cloned (Genscript) into pET26B(+) vectors (Novagen) with C-terminal His₆ tags.

Recombinant IL-38 variants were expressed in BL21-CodonPlus(DE3)-RIL cells (Stratagene) by 0.5 mM IPTG induction (OD₆₀₀ 0.8) overnight at 18° C. Cells expressing IL-38 variants were lysed by sonication in 50 mM Tris-HCl (pH 8.0), 500 mM NaCl, 3 mM β-mercaptoethanol, and 30 mM imidazole. Cells were clarified by centrifugation, filtered through a 0.8 μm membrane, and bound to Ni-NTA resin (Qiagen) for 1 hour at 4° C. on a rotating platform. The resin was washed with 300 ml of 50 mM Tris-HCl (pH 8.0), 500 mM NaCl, 3 mM β-mercaptoethanol, and 30 mM imidazole. The protein was eluted from the Ni-NTA resin (Qiagen) in a buffer containing 50 mM Tris-HCl (pH 8.0), 500 mM NaCl, 3 mM β-mercaptoethanol, and 400 mM imidazole. Protein was further purified on a Superdex 75 Increase 10/300 GL column (Cytiva Life Sciences) in 10 mM HEPES (pH 7.2), 150 mM NaCl, and 2 mM DTT.

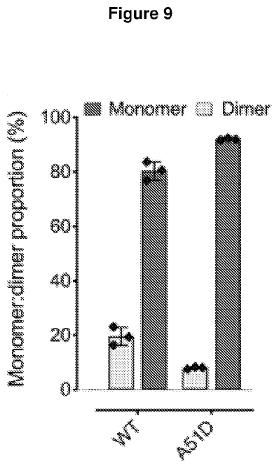

The number and elution volume of the elution peaks are indicative of whether the size of the recombinant protein corresponds to monomeric or dimeric IL-38 conformations or both conformations. The proportion of monomer: dimer for each mutant (FIGS. 9 and 11) were calculated by comparing the area under the monomer and dimer peaks from three individual purifications.

The homodimer interface can be can modulated and form a stable locked dimer (ΔT53), which allows testing of the functional properties of an IL-38 homodimer. Various other mutations potentiate or hinder homodimer formation. Mutations that result in a reduced capacity to form a homodimer, or that have an increased capacity to form a monomer, including substitution mutations at position 51 (for example A51D), position 53 (for example, T53N), position 55 (for example V55Q or V55T) (FIG. 11).

### Example 5: Analytical SEC Analysis of IL-38 Variants Upon Extended Incubation

WT IL-38 forms ˜20% dimer upon purification, ˜80% monomer (FIG. 9).

To examine the monomer-dimer equilibrium upon extended incubation, indicated IL-38 variants (residues 3-152, C-terminal His₆ tag) were incubated at 0.165 mg/ml and 37° C. for 16 hours in Sorensen's phosphate buffer (0.13 M, pH 7.0) with 5% (v/v) glycerol. The monomer: dimer proportion was examined by analytical SEC on a Superdex 75 Increase 10/300 GL column (Cytiva Life Sciences).

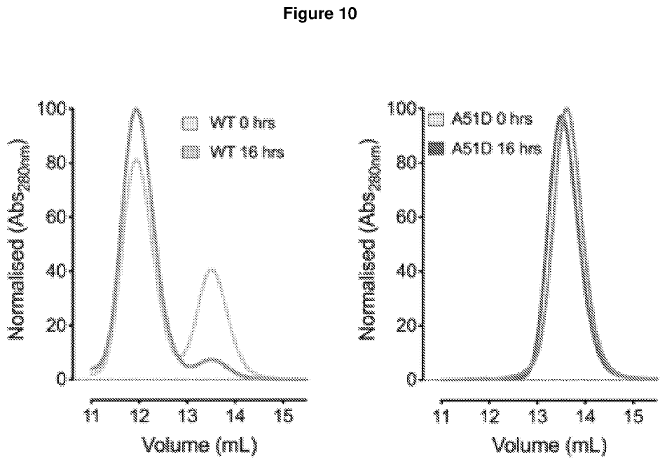

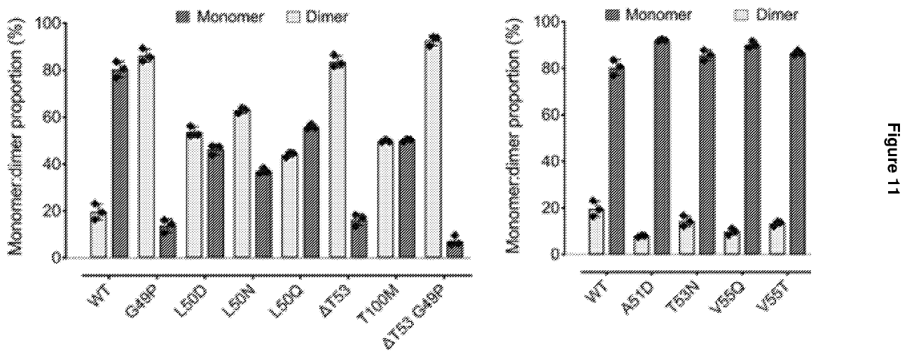

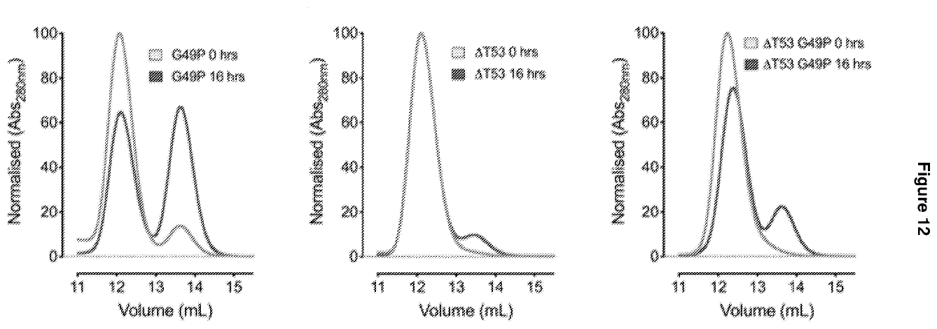

As shown in FIG. 10, WT IL-38 exists in a slow equilibrium between the two states. IL-38 (A51D) is a more stable monomer.

The locked dimer (ΔT53) variant is stable as a dimer (e.g. versus G49P upon prolonged incubation at 37 deg for 16 hrs) (FIG. 12).

### Example 6: Structure of Homodimer Locked IL-38 (ΔT53)

The inventors solved the structure of the locked dimer IL-38 (ΔT53) to 1.7 Å. IL-38 (ΔT53) crystals were grown at 20° C. by sitting drop vapour diffusion in 25% (w/v) polyethylene glycol 3350, 0.1 M HEPES pH 7.5, and 0.2 M MgCl₂ at a 1:1 drop ratio. Crystals were flash cooled in liquid nitrogen in mother liquor containing 25% (v/v) glycerol. X-ray data were collected at the MX2 beamline (microfocus) of the Australian Synchrotron at a wavelength of 0.9573. Data were processed and scaled using XDS and programs within the CCP4 suite. The structure was solved by molecular replacement using monomeric IL-38 (PDB code 5BOW) as the search model in PHENIX using the Phaser program. The initial structure was rebuilt manually in COOT and iterative cycles of refinement were carried out using Phenix Refine with local rebuilding in COOT, resulting in a model with an R-factor of 16.64% (R_{free} of 19.13%) and excellent geometry (Table 3). The structure had no Ramachandran outliers with 97.24% of residues in favored regions and a final MolProbity clash score of 1.73.

This structure and confirms the location of the dimer hinge-region and interface shown in FIG. 5A.

### Example 7: Generation of Monomer IL-38 Fusions

Codon-optimized sequences for IL-38-Fc were synthesized and cloned (Genscript) into pcDNA3.1 (+) (ThermoFisher Scientific). Codon-optimized sequences for Fc (Genscript) were separately synthesized and cloned into pcDNA3.1 (+) (ThermoFisher Scientific).

Indicated variants of the IL-38 moiety fused to the fragment crystallizable region (Fc region) of a human antibody sequence were co-expressed with a non-fused Fc region of a human antibody sequence to form a Fc-fusion protein with a single IL-38 moiety. An equimolar amount of each expression vector was transiently transfected in Expi293F™ cells (suspension-adapted Human Embryonic Kidney (HEK) cells, ThermoFisher Scientific) using polyethyleneimine (PEI) at a 4:1 PEI to DNA ratio. IL-38-Fc variants were cultivated in Expi293TM Expression Medium and expressed for 4-5 days in a shaker incubator at 37° C., a speed of 110 rpm, and in an atmosphere supplemented with 5% CO₂.

Cell media was harvested by centrifugation at 561 g for 10 minutes and filtered through a 0.45 μm filter membrane. Phosphate-buffered saline (PBS) was added to the media to a 1× final concentration (0.137 M NaCl, 0.0027 M KCl, 0.01 M Na₂HPO₄, 0.0018 M KH₂PO4) and passed through a HiTrap Protein G HP column (Cytiva Life Sciences). The column was washed with PBS, and IL-38-Fc eluted with 0.1 M Glycine (pH 2.74) and immediately diluted in 1 M Tris (pH 9.0). Eluted protein was concentrated in a 10 kilodalton molecular weight cut-off Amicon Ultra Centrifugal Filter Unit (Millipore) and loaded onto a Superdex 200 10/300 size exclusion column (Cytiva Life Sciences) equilibrated in Dulbecco's phosphate-buffered saline (ThermoFisher Scientific). Purified IL-38-Fc was pooled, sterile filtered through 0.2 μm membrane, snap-frozen in liquid nitrogen, and stored at −80° C.

IL-38 is an intracellular protein that contains no disulfide bonds but six unpaired cysteines. Therefore, there is the potential for major issues upon secreting (via ER) intracellular proteins (e.g. IL-38) as Fc/Albumin fusions. Unpaired cysteines form unwanted disulfides leading to aggregation. All cysteines were removed and test co-expression as fusion.

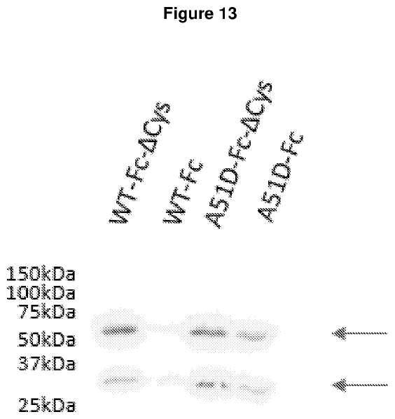

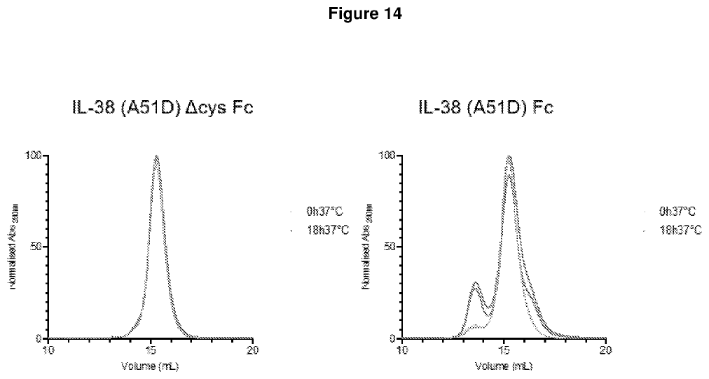

IL-38 (WT) cannot be produced with cysteines as shown in FIG. 12. Surprisingly, A51D-Fc can be produced but with significantly reduced expression (FIG. 13). Not only does removal of the cysteines in the context of an A51D fusion result in increased yield, but it results in increased stability of the Fc fusion (FIG. 14).

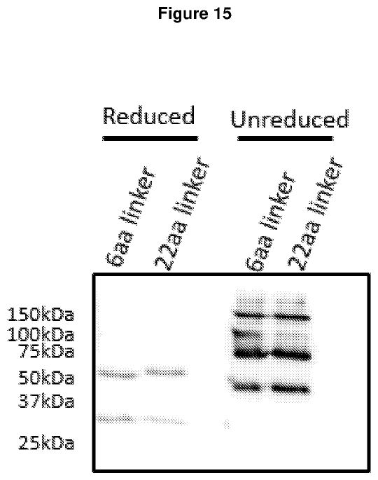

IL-38 fusions with different length linkers between the Fc and IL-38 polypeptide were produced (FIG. 15).

Unless otherwise specified, the Fc fusion proteins referred to herein have had all 6 cysteines removed.

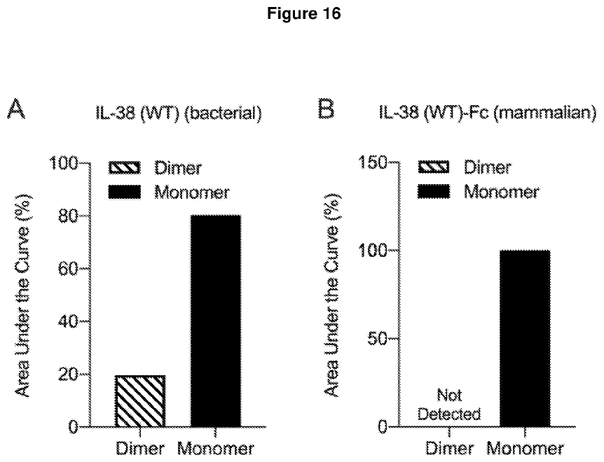

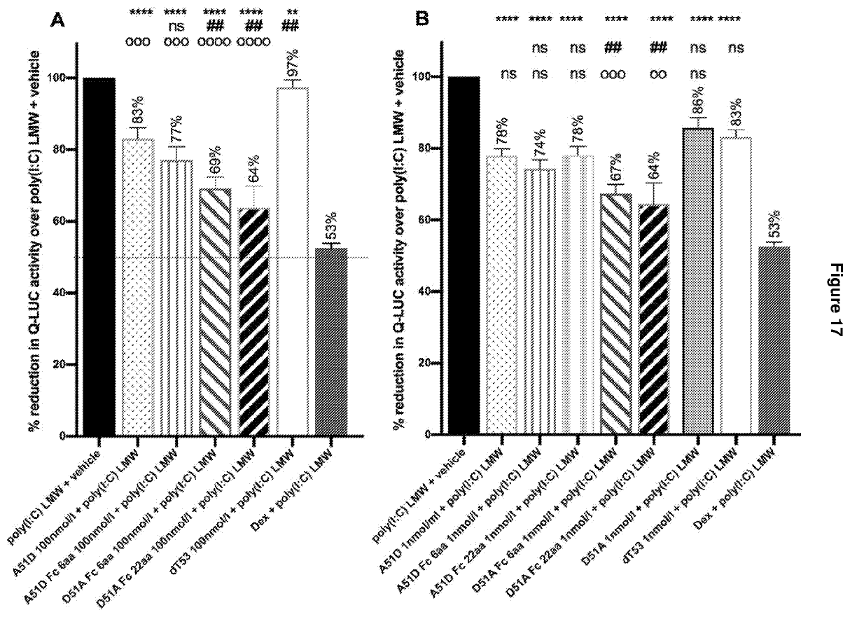

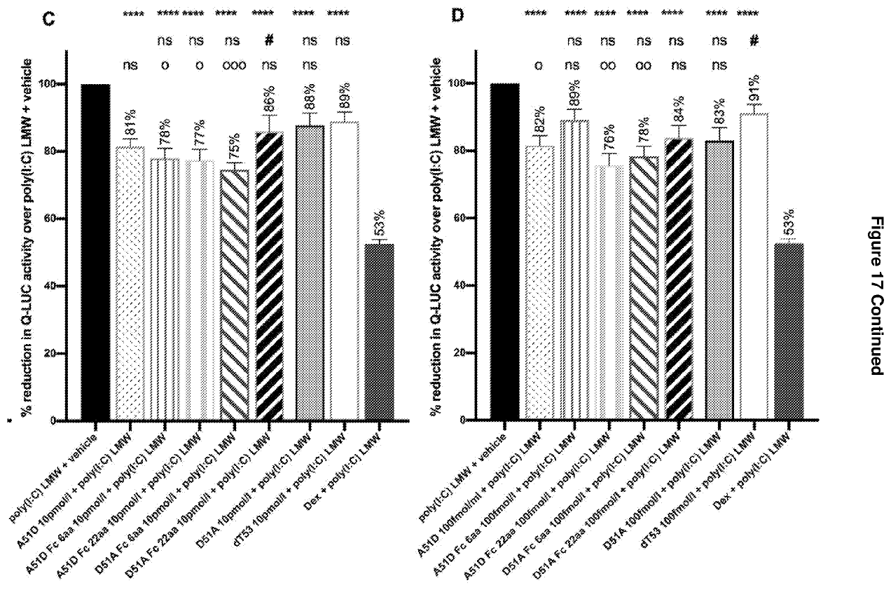

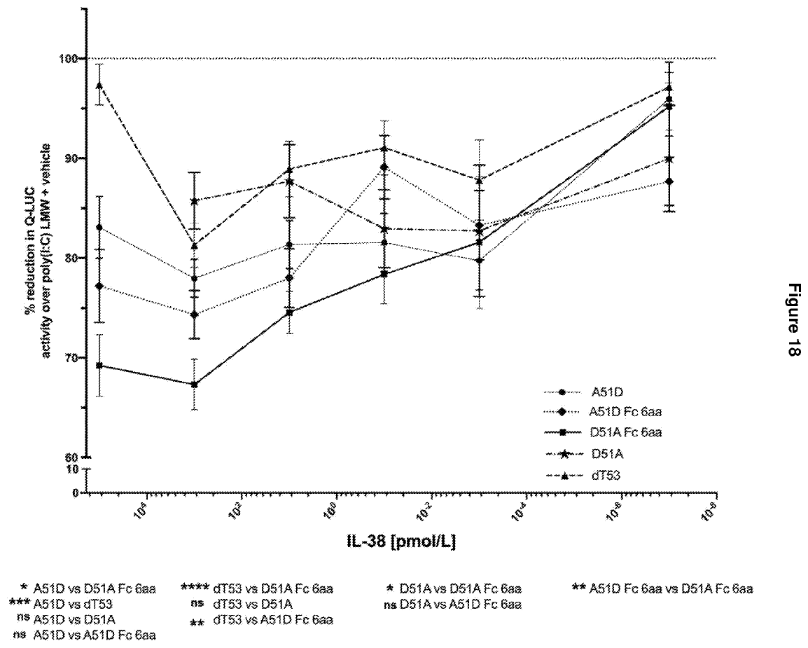

Fusion of IL-38 with the Fc protects IL-38 from homodimer formation (FIG. 16).

### Example 5: In Vitro Functional Analysis of Variant IL-38 Polypeptides

Measurement of IRF3/7 Activation by Quanti-Luc in A549-Dual Cells from InvivoGen

6 μl of A549 Dual cell supernatants or plain stimulation media (background control) and 15 μl of QUANTI-Luc™ solution (InvivoGen) were added to a 384-well plate (Opti-plate-384, PerkinElmer, #6007290) and immediately measured as per manufacturer's instructions. After background subtraction, the %-change in luciferase activity of IL-38 variants (1 nm/l)+Poly (I:C) treated conditions over poly (I:C)+vehicle-treated cells were calculated (absorbance of cells treated with poly (I:C)+vehicle=100%).

Culture supernatants were then subjected to IL-6 (hIL-6 AlphaLISA, Perkin Elmer, Cat #AL223F) and RANTES (hRANTES ELISA Cat #DY278-05, R&D Systems) analysis according to the manufacturer's instructions. To account for any variability in IL-6 and RANTES responses, data were graphed as percent changes rather than as absolute concentrations. The %-change in either IL-6 or RANTES protein expression in IL-38+Poly (I:C) treated conditions over poly (I:C)+vehicle-treated cells were calculated (absorbance of cells treated with poly (I:C)+vehicle=100%).

### RNA Isolation and Detection of Gene Expression in A549 Dual Cells

According to the manufacturer's instructions, total RNA was isolated using the RNA Mini Kit (Bioline) and quantified with a NanoDrop (ND-1000) spectrophotometer (Thermo Fisher Scientific). Reverse transcription was performed using the Tetro cDNA Synthesis kit (Bioline). TaqMan real-time PCRs (TaqMan Gene Expression Master Mix and TaqMan Gene Expression Assay [IFNL2/3: Hs04193049_gH, IFNB1: Hs01077958_s1, ACTB: Hs01060665_g1], Thermo Fisher Scientific) and SYBR Green real-time PCRs (Power SYBR Green PCR Master Mix, Thermo Fisher Scientific and oligonucleotide primers [forward/reverse], Gene Works for ACTB [5′-GTC ATT CCA AAT ATG AGA TGC GT-3′/5′-GCT ATC ACC TCC CCT GTG TG-3′], ISG15 [5′-GCG AAC TCA TCT TTG CCA GT-3′/5′-AGC ATC TTC ACC GTC AGG TC-3′] and CXCL10 [5′-TTC CTG CAA GCC ATT TTG T-3′ /5′-TTC TTG ATG GCC TTC GAT TC-3′]) were performed according to the manufacturer's instructions on the QuantStudio 6 RT-PCR system (Thermo Fisher Scientific). Samples and no template controls for each gene master mix were run in duplicate. The cycling conditions were as follows: initial denaturation at 95° C. for 10 min, followed by 40 cycles of denaturation at 95° C. for 15 s and annealing/elongation at 60° C. for 1 min. For SYBR PCR these cycles were followed by a melt-curve analysis, 95° C. for 15 seconds, 60° C. for 15 seconds, followed by 95° C. for 15 seconds. Relative expression quantification was calculated according to Pfaffl's method. The fold-changes for all gene expression calculations were normalized to ACTB mRNA.

As shown in FIG. 17A-D, the locked dimer ΔT53 IL-38 variant exhibited only marginal reduction in poly (I:C)-induced IRF3/7. This shows that dimerization strongly reduces the function of IL-38. Consequently, to generate a potent therapeutic based on the anti-inflammatory function of IL-38, the IL-38 should have a reduced capacity to form a dimer and the composition administered should contain an increased proportion of monomer (or a reduced proportion of dimer).

Surprisingly, WT (D51A) IL-38 Fc fusion was more potent than A51D IL-38 and A51D IL-38 Fc fusion (FIGS. 17 and 18). This is surprising as the D51A (non-fused) IL-38 was slightly less potent than A51D (non-fused) IL-38. Importantly, the efficacy of the IL-38 Fc fusions was approaching that of dexamethasone. The length of the linker did not have any significant impact of the efficacy of the IL-38 Fc fusions.

All of the IL-38 variants tested, whether part of an Fc fusion or not, potently reduced poly (I:C)-induced IRF3/7.

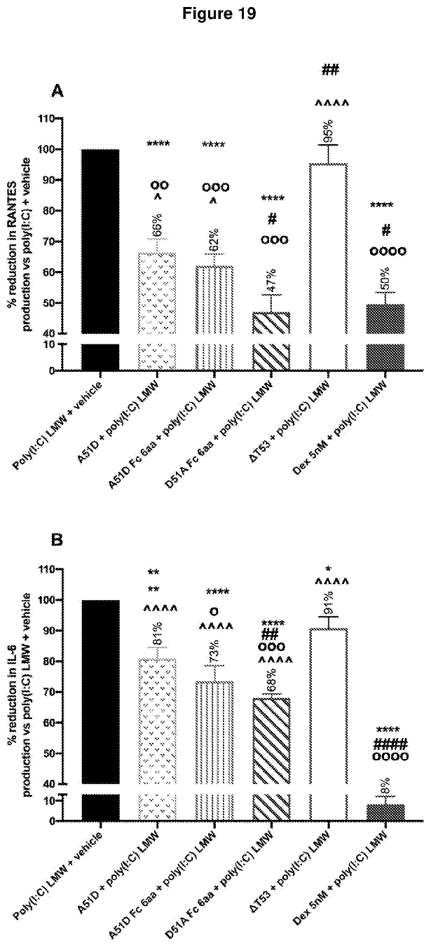

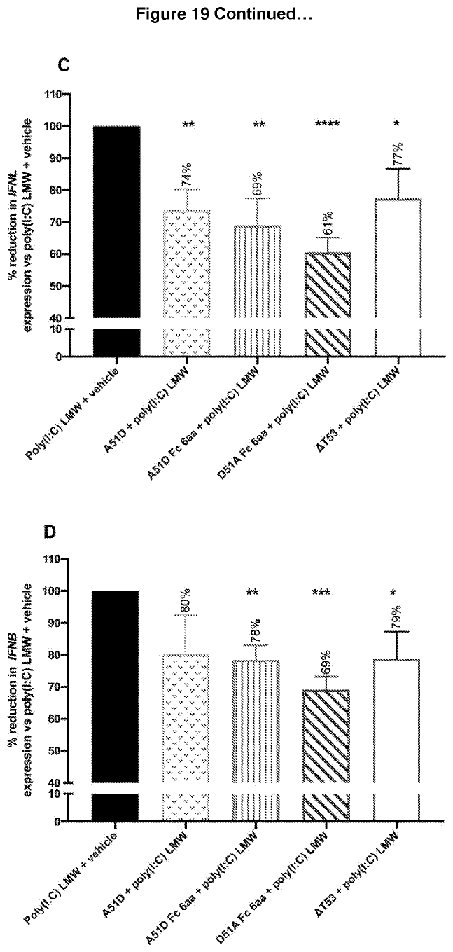

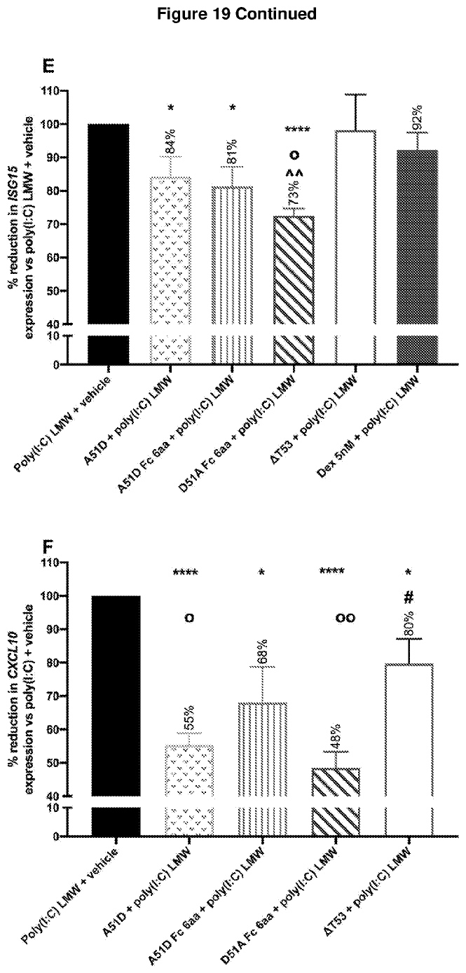

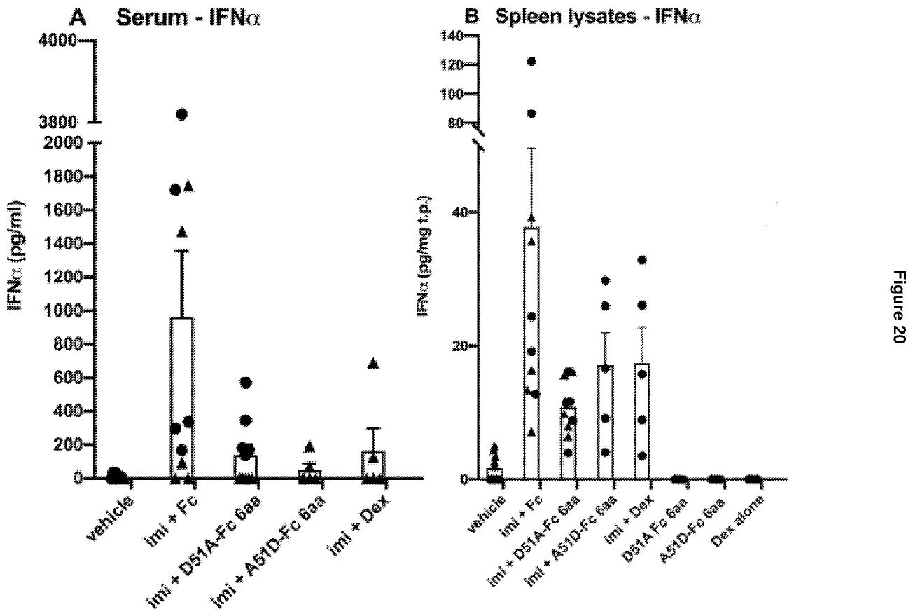

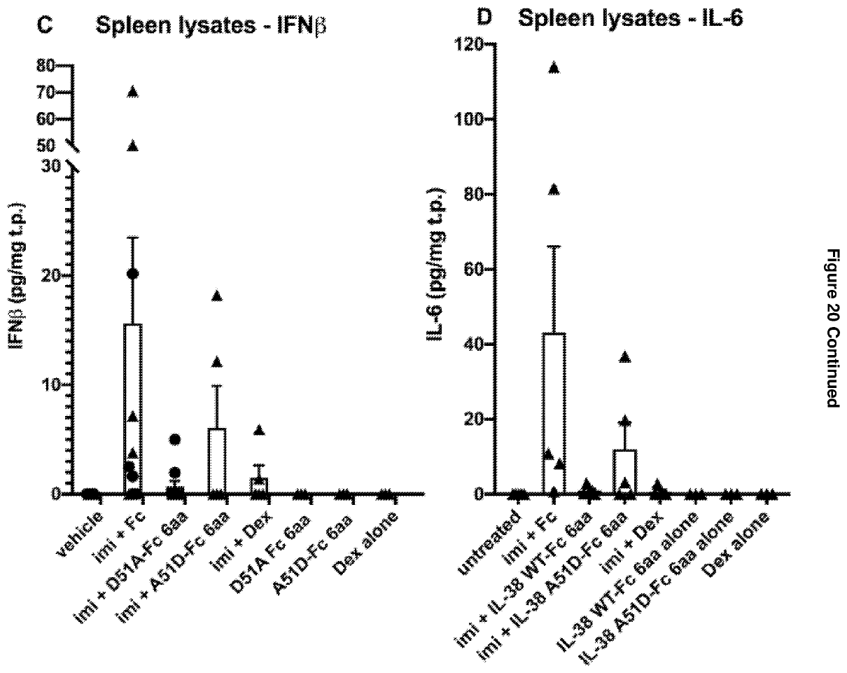

Further, WT (D51A) IL-38 Fc was as potent as dexamethasone at suppressing RANTES protein expression (FIG. 19A) and was more potent than A51D and A51D IL-38 Fc fusion. WT (D51A) IL-38 Fc was also the most potent IL-38 variant at suppressing IL-6 protein expression (FIG. 19B). WT (D51A) IL-38 Fc was more potent than dexamethasone at suppressing ISG15 mRNA expression level (FIG. 19E).

As shown in FIG. 19A-F, dimerization strongly reduces the capacity of IL-38 to suppress inflammatory mediators.

The IL-38 variants-mutants and fusions-described herein potently reduce innate immune sensor-triggered type I IFN pathways, with affected mediators including (but not limited to) IRF3&7, IFNalpha, IFNbeta, IFNlambda, RANTES, CXCL10; however, the NF-kappa B pathway is also suppressed, albeit less efficiently.

When targeting the type I IFN pathway, the IL-38 mutants and fusions are as efficient, and in some instances better, at suppressing inflammation than the clinical gold-standard dexamethasone (a powerful, but side-effect-prone corticosteroid)-a highly unusual and highly desirable property.

### Example 6: In Vivo Functional Analysis of Variant IL-38 Polypeptides

All experiments involving mice were approved by Monash University's Animal Ethics Committee. Eight to twelve-week-old male C57Bl/6J mice were injected subcutaneously (s.c.) with saline, Fc control, dexamethasone (300 ng/g) or either D51A-Fc, A51D-Fc (40 μg/kg IL-38 normalized to molecular weight). One hour later mice received and intraperitoneal (i.p.) injection with either saline or imiquimod (5 μg/g bodyweight). Room temperature and humidity were monitored continuously.

Six hours after imiquimod injections, mice were anesthetized, and serum was obtained by cardiac bleeding into BD serum tubes. Spleens were harvested and spleenlysates were prepared for cytokine analysis as previously described in Nold-Petry et al Nature Immunology 2015. Serum and spleen cytokine levels were determined by either Alphalisa for murine IL-6 (PerkinElmer, Cat #AL504F) or by ELISA for murine IFNalpha (#PBL-42120) and beta (#PBL-42400), according to the manufacturers' instructions.

Functional testing of fusion proteins in mechanistic model for disease relevant pathway TLR7 stimulation. IL-38 D51A-Fc presents with activity in a mechanistic murine model of disease (imiquimod (imi) i.p. 5 μg/g). S.c. administration of D51A-Fc and A51D Fc-fusion was tested at 40 μg/kg normalized to molecular weight 1h prior to imiquimod i.p. injections. Serum and spleens were harvested at 6h after imiquimod injection and IFNalpha, IFNbeta and IL-6 were determined by ELISA. Readouts in spleenlysates were normalized to total protein. Depicted are two independent experiments, n=5-10/group.

As shown in FIG. 20, both IL-38 Fc fusions were as potent or more potent than dexamethasone at reducing imiquimod driven increase in inflammatory mediators. This highlights the in vivo efficacy and surprisingly potent anti-inflammatory functions of IL-38 Fc fusions of the invention.

### Example 7: Cysteine Variants

Purification of interleukin-38 (IL-38) as a fragment crystallizable (Fc)-fusion protein from mammalian cell culture required the mutation of specific cysteine residues within the IL-38 sequence. The combination of cysteine-mutations was determined experimentally. Briefly, each indicated variant was cloned and expressed in Expi293 cells. Then, the protein was purified sequentially using a Protein G affinity column (to the Fc region) followed by size exclusion chromatography. The success of the purification was monitored by the presence of a single peak on size exclusion chromatography and pure bands by SDS-PAGE and Coomassie staining.
The inventors discovered that the most consistent yield was obtained for the IL-38-Fc variant with all six IL-38 cysteine residues mutated in the following combination (C70V, C37S, C38R, C43A, C67A, C123S). The specific mutants were designed from analysis of the IL-38 crystal structure.
Removal of only five cysteine residues also yielded high-quality IL-38-Fc. However, in every case, C70V and C37S were required to be mutated.
Mutation of only four cysteines reduced both the yield and quality of the protein.
Only low-quality, or no protein, was purified when only three or fewer cysteines were mutated.
All cysteine variants were trialled as a “knobs-into-holes” IgG1-Fc fusion.

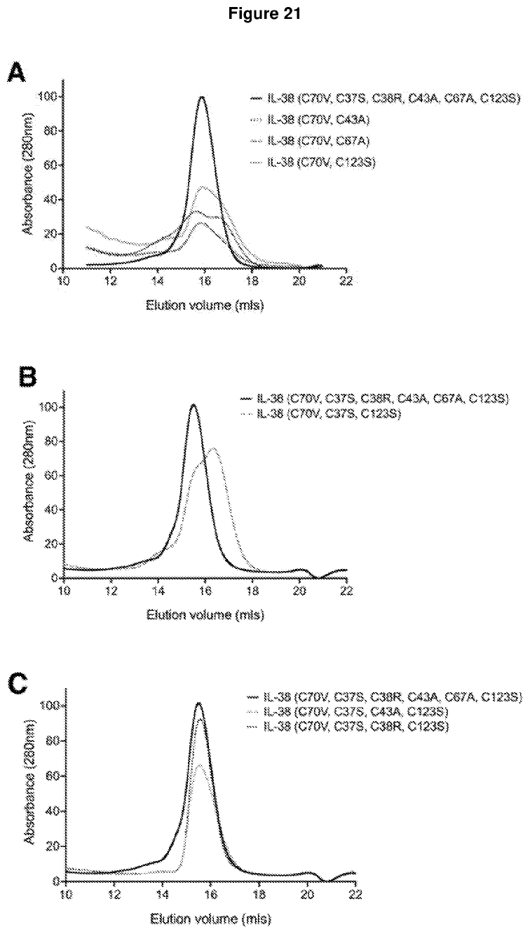

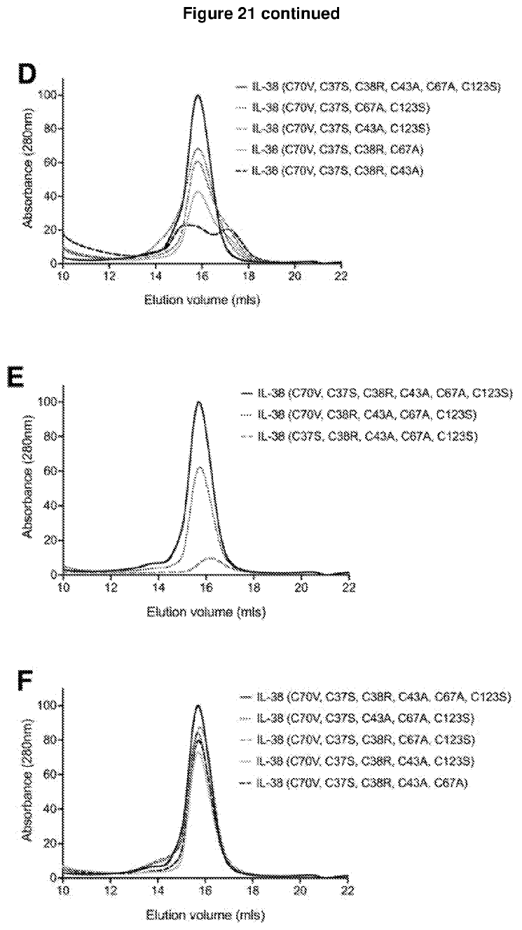

These data are summarised in Table 4 with representative size-exclusion chromatograms of selected variants displayed in FIGS. 21A-F.

TABLE 4
Summary of IL-38-Fc purification

| | | Figure |
|---|---|---|
| IL-38-Fc variant | Yield | Reference |
| IL-38 | − | − |
| IL-38 (C70V) | − | − |
| IL-38 (C37S) | − | − |
| IL-38 (C38R) | − | − |
| IL-38 (C43A) | − | − |
| IL-38 (C67A) | − | − |
| IL-38 (C123S) | − | − |
| IL-38 (C70V, C67A) | − | FIG. 21A |
| IL-38 (C70V, C123S) | − | FIG. 21A |
| IL-38 (C70V, C43A) | − | FIG. 21A |
| IL-38 (C70V, C38R) | − | − |
| IL-38 (C70V, C37S) | − | − |
| IL-38 (C70V, C43A, C67A) | − | − |
| IL-38 (C70V, C43A, C123S) | − | − |
| IL-38 (C70V, C38R, C43A) | + | − |
| IL-38 (C70V, C37S, C123S) | + | FIG. 21B |
| IL-38 (C70V, C37S, C38R, C67A) | + | FIG. 21D |
| IL-38 (C70V, C37S, C67A, C123S) | + | FIG. 21D |
| IL-38 (C70V, C37S, C38R, C43A) | + | FIG. 21D |
| IL-38 (C70V, C37S, C38R, C123S) | ++ | FIG. 21C |
| IL-38 (C70V, C37S, C43A, C67A) | ++ | FIG. 21C |
| IL-38 (C70V, C37S, C43A, C123S) | + | FIG. 21D |
| IL-38 (C37S, C38R, C43A, C67A, C123S) | + | FIG. 21E |
| IL-38 (C70V, C38R, C43A, C67A, C123S) | ++ | FIG. 21E |
| IL-38 (C70V, C37S, C43A, C67A, C123S) | +++ | FIG. 21F |
| IL-38 (C70V, C37S, C38R, C67A, C123S) | +++ | FIG. 21F |
| IL-38 (C70V, C37S, C38R, C43A, C123S) | +++ | FIG. 21F |
| IL-38 (C70V, C37S, C38R, C43A, C67A) | +++ | FIG. 21F |
| IL-38 (C70V, C37S, C38R, C43A, C67A, C123S) | +++ | FIG. 21A-F |
| IL-38 (C70V, C37S, C38R, C43A, C67A, C123S, A51D) | +++ | − |

(−) no or low-quality protein purified
(+) soluble protein purified but of low quality
(++) good yield and purity
(+++) maximum yield and purity

### Example 8: N Terminal Variants

Specific truncation of the N-terminus of IL-1 cytokine family members may regulate the activity of IL-1 family members. As such, it is important to optimise the amino-terminal residue of the IL-38 chain to obtain the highest anti-inflammatory activity, stability, or yield. The inventors trialled six IL-38 N-terminal variants (Table 5). The inventors found the specific N-terminal residue of the IL-38 chain had a substantial effect on the purification yield of the IL-38-Fc.

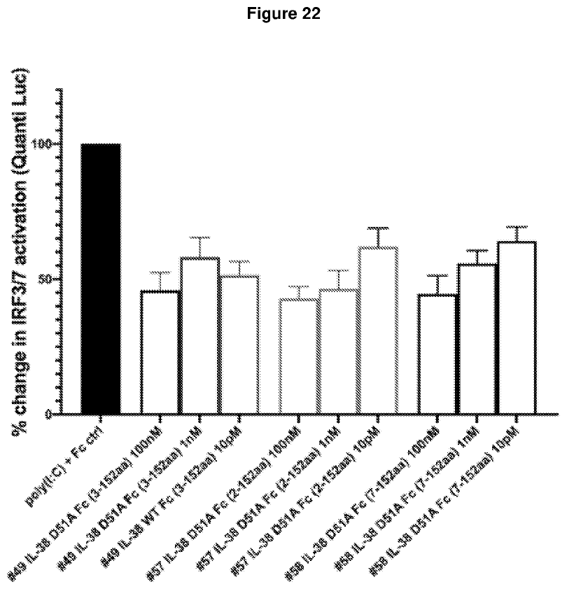

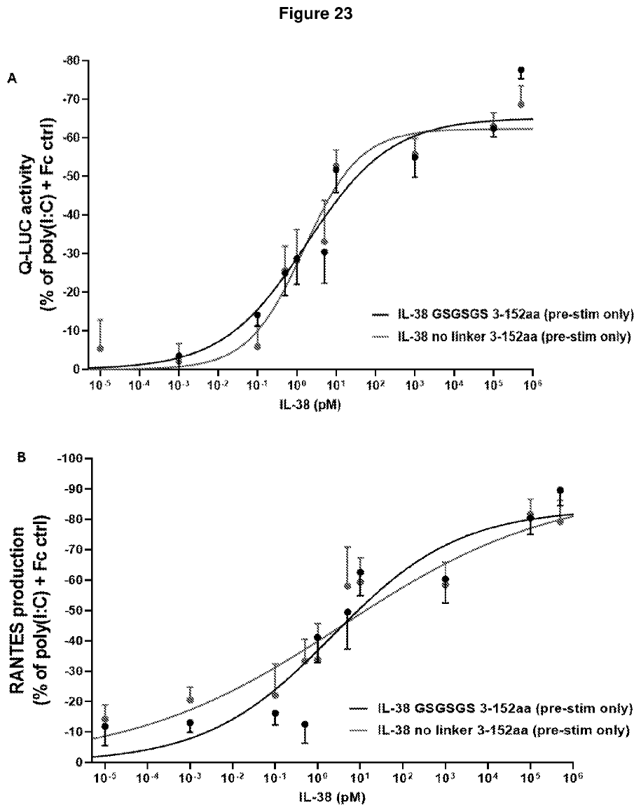

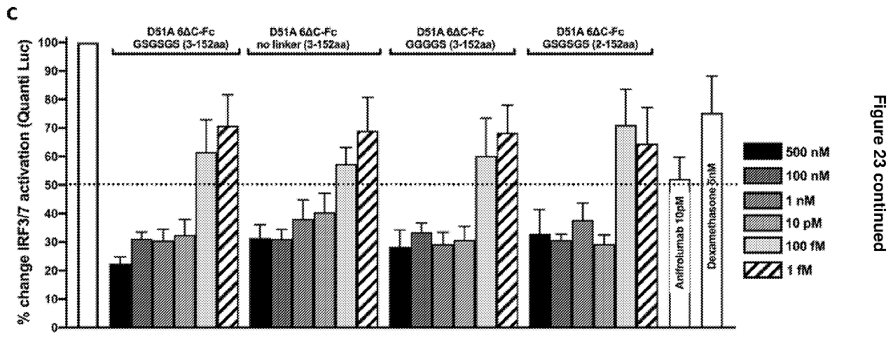

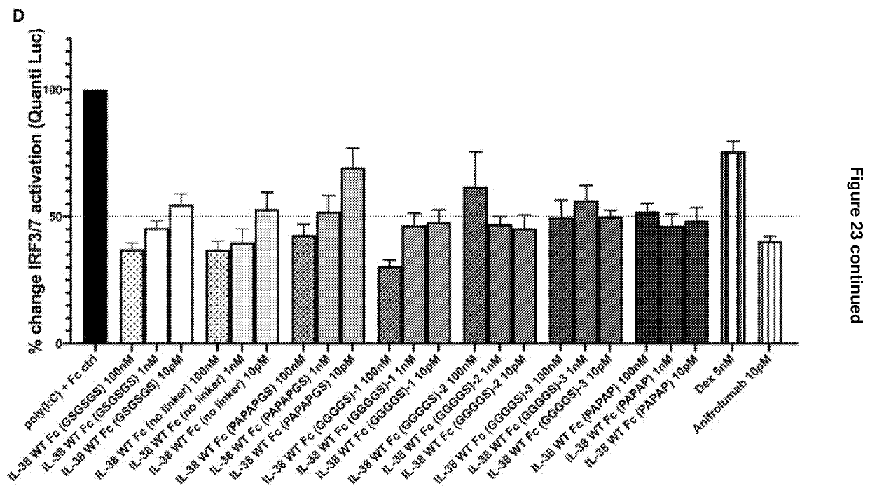

TABLE 5
Purification and stability of indicated N-terminal IL-38-Fc variants.

| IL-38 chain residues | Yield | Stability | Bioactivity |
|---|---|---|---|
| 1-152 | +++ | 66.5° C. | |
| 2-152 | ++ | − | FIG. 22 and 23C |
| 3-152 | +++ | 66.0° C. | FIG. 22 and 23C |
| 5-152 | − | − | |
| 7-152 | + | 66.4° C. | FIG. 22 |
| 20-152 | − | − | |
| 21-152 | − | − | |

(−) no yield
(+) low yield
(++) moderate yield
(+++) maximum yield

Indicated IL-38 chains differing in their N-terminus were expressed and purified from Expi293 cells. The relative purification yield is indicated between the N-terminal variants. The stability of the purified product was monitored by nano differential scanning fluorimetry and is expressed as the apparent melting temperature (Tm). N-terminal variants (except 2-152) were trialled as “knobs-into-holes” IgG1-Fc fusions utilising the IL-38 (C70V, C37S, C38R, C43A, C67A, C123S) sequence. The 2-152 N-terminal variant was trialled as “knobs-into-holes” IgG₁-Fc fusions utilising the IL-38 (C2A, C70V, C37S, C38R, C43A, C67A, C123S) sequence.

### Example 9: Linker Variants

Modification of the amino-acids (linker) that separate the IL-38 moiety from the Fc may alter the expression, stability, or activity of the IL-38-Fc. The inventors trialled expression, purification, and stability of the linker regions indicated in Table 6.

TABLE 6
Purification and stability of indicated linker variants

| Linker | Yield | Stability (Tₘ) | Bioactivity |
|---|---|---|---|
| No linker | +++ | 65.1° C. | FIG. 3A |
| GSGSGS | +++ | 66.0° C. | FIG. 3B |
| (GGGGS)₁ | + | 66.1° C. | FIG. 3C, D |
| (GGGGS)₂ | + | 66.4° C. | FIG. 3D |
| (GGGGS)₃ | + | 66.4° C. | FIG. 3D |
| PAPAP | ++ | 66.1° C. | FIG. 3D |

(+) low yield
(++) moderate yield
(+++) maximum yield

Indicated linker variants were expressed and purified from Expi293 cells. The relative purification yield is indicated between the linker variants. The stability of the purified product was monitored by nano differential scanning fluorimetry and is expressed as the apparent melting temperature (Tₘ). Although the expression and purification yields are substantially different between the linker variants, the stability (as monitored by Tₘ) of the variants is comparable. All N-terminal linker variants were trialled as “knobs-into-holes” IgG1-Fc fusions utilising the IL-38 (C70V, C37S, C38R, C43A, C67A, C123S) sequence.

### Example 10: IgG1 Variants

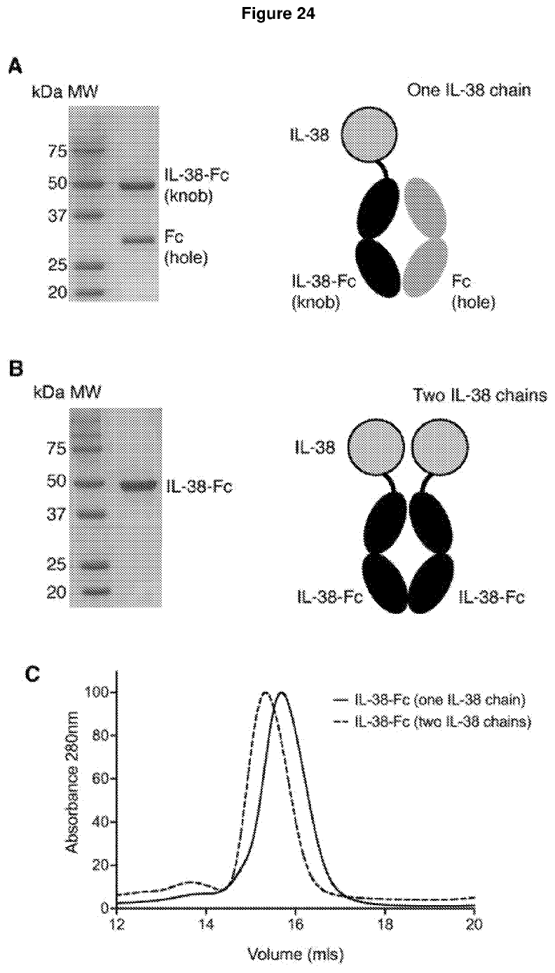

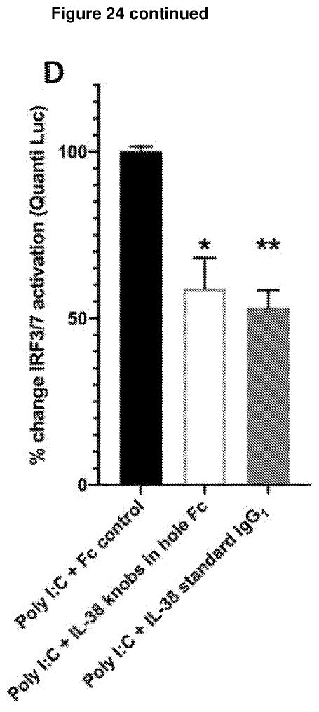

Two Fc variants of the human IgG1 sequence were employed in the purification of IL-38-Fc. Cysteine, linker, and N-terminal variants were trialled and optimised using a “knobs-into-holes” IgG1 sequence that enables expression and purification of an IL-38-Fc with only one copy of IL-38 per molecule (FIG. 24A). The inventors also trialled the expression of IL-38-Fc (C70V, C37S, C38R, C43A, C67A, C123S) as a standard Fc-fusion protein that enables expression and purification of an IL-38-Fc with two IL-38 copies per molecule (FIG. 24B). Size exclusion chromatography of each molecule demonstrates that each molecule has excellent final purity (FIG. 24C) and bioactivity in Poly I: C stimulated A549Dual cells (FIG. 24D).

1. A fusion protein comprising an IL-38 polypeptide and an Fc region of an antibody.

2. The fusion protein of claim 1, wherein the fusion protein is capable of forming a heterodimeric molecule that comprises a single IL-38 polypeptide amino acid sequence.

3. The fusion protein of claim 1 or 2, wherein the Fc region comprises one or more amino acid substitutions for enabling the fusion protein to form a heterodimer with an Fc region of an antibody that does not comprise an IL-38 polypeptide fused thereto.

4. The fusion protein of any one of claims 1 to 3, wherein the Fc region comprises at least one substitution or modification to provide for a knob-into-hole dimerisation domain in the Fc region.

5. The fusion protein of any one of claims 1 to 3, wherein the Fc region comprises the amino acid sequence as set forth in SEQ ID NO: 20 or SEQ ID NO: 9, or a sequence at least 80% identical thereto.

6. The fusion protein of any one of claims 1 to 5, wherein the IL-38 polypeptide has a substitution of one or more cysteine residues at, or at a position equivalent to, 37, 38, 43, 67, 70 and 123 of SEQ ID NO: 1 or a functional variant thereof.

7. The fusion protein of claim 6, wherein the IL-38 polypeptide comprises a substitution of two or more cysteine residues at, or at a position equivalent to, residues 37, 38, 43, 67, 70 and 123 of SEQ ID NO: 1 or a functional variant thereof.

8. The fusion protein of claim 6, wherein the IL-38 polypeptide comprises a substitution of three or more cysteine residues at, or at a position equivalent to, residues 37, 38, 43, 67, 70 and 123 of SEQ ID NO: 1 or a functional variant thereof.

9. The fusion protein of claim 6, wherein the IL-38 polypeptide comprises a substitution of at least 4, at least 5 or all of the cysteine at residues at, or at positions equivalent to residues 37, 38, 43, 67, 70 and 123 of SEQ ID NO: 1 or a functional variant thereof.

10. The fusion protein of claim 6, wherein the IL-38 polypeptide has a substitution of the cysteine residues at, or at a position equivalent to, at least residues 37 and 70 of SEQ ID NO: 1 or a functional variant thereof.

11. The fusion protein of claim 6, wherein the IL-38 polypeptide comprises a substitution of at least the cysteine residues at, or at a position equivalent to, residue 37, 43, 67 and 70 of SEQ ID NO: 1 or a functional variant thereof.

12. The fusion protein of claim 6, wherein the IL-38 polypeptide comprises a substitution of at least the cysteine residues at, or at a position equivalent to, 37, 38, 67 and 70 of SEQ ID NO: 1 or a functional variant thereof.

13. The fusion protein of claim 6, wherein the IL-38 polypeptide comprises a substitution of at least the cysteine residues at, or at a position equivalent to, 37, 67, 70 and 123 of SEQ ID NO: 1 or a functional variant thereof.

14. The fusion protein of claim 6, wherein the IL-38 polypeptide comprises a substitution of at least the cysteine residues at, or at a position equivalent to, 37, 38, 70 and 123 of SEQ ID NO: 1 or a functional variant thereof.

15. The fusion protein of claim 6, wherein the IL-38 polypeptide comprises a substitution of at least the cysteine residues at, or at a position equivalent to, 37, 43, 67 and 70 of SEQ ID NO: 1 or a functional variant thereof.

16. The fusion protein of claim 6, wherein the IL-38 polypeptide comprises a substitution of at least the cysteine residues at, or at a position equivalent to:
a) residues 37, 43, 67, 70 and 123 of SEQ ID NO: 1 or a functional variant thereof, or
b) residues 37, 38, 67, 70 and 123 of SEQ ID NO: 1 or a functional variant thereof, or
c) residues 37, 38, 43, 70 and 123 of SEQ ID NO: 1 or a functional variant thereof; or
d) residues 37, 38, 43, 67, and 70 of SEQ ID NO: 1 or a functional variant thereof.

17. The fusion protein of any one of claims 1 to 16, wherein the IL-38 polypeptide comprises:
an amino acid residue selected from the group consisting of serine, threonine, glycine, alanine, arginine, valine, glutamine and asparagine, at, or at a position equivalent to, residue 37 of SEQ ID NO: 1 or a functional variant thereof;
an amino acid residue selected from the group consisting of serine, threonine, glycine, alanine, arginine, valine, glutamine and asparagine, at, or at position equivalent to, residue 38 of SEQ ID NO: 1 or a functional variant thereof;
an amino acid residue selected from the group consisting of serine, threonine, glycine, alanine, arginine, valine, glutamine and asparagine, at, or at position equivalent to, residue 43 of SEQ ID NO: 1 or a functional variant thereof;
an amino acid residue selected from the group consisting of serine, threonine, glycine, alanine, arginine, valine, glutamine and asparagine, at, or at position equivalent to, residue 67 of SEQ ID NO: 1 or a functional variant thereof;
an amino acid residue selected from the group consisting of serine, threonine, glycine, alanine, arginine, valine, glutamine and asparagine, at, or at position equivalent to, residue 70 of SEQ ID NO: 1 or a functional variant thereof; and/or
an amino acid residue selected from the group consisting of serine, threonine, glycine, alanine, arginine, valine, glutamine and asparagine, at, or at position equivalent to, residue 123 of SEQ ID NO: 1 or a functional variant thereof.

18. The fusion protein of claim 6, wherein the substitution of the cysteine residues at positions, or positions equivalent to 37, 38 and/or 123 of SEQ ID NO: 1, or a functional variant thereof, comprises a substitution to a hydrophilic amino acid residue (such as arginine, lysine, histidine, aspartic acid, glutamic acid, serine, threonine, tyrosine, asparagine or glutamine).

19. The fusion protein of claim 17, wherein the substitution of cysteine residue at positions, or positions equivalent to 37, 38 and/or 123 is to a serine or arginine residue.

20. The fusion protein of claim 19, wherein the substitution at residue 37 and/or residue 123 is to a serine residue, and/or the substitution at residue 38 is to an arginine.

21. The fusion protein of claim 17, wherein the substitution of the cysteine residues at positions, or positions equivalent to 43, 67 and 70 of SEQ ID NO: 1, or a functional variant thereof, comprises a substitution to a hydrophobic amino acid residue (such as valine, leucine, isoleucine, phenylalanine, methionine, glycine or alanine).

22. The fusion protein of claim 21, wherein the substitution of the cysteine residue at positions, or positions equivalent to residues 43, 67 and/or 70 of SEQ ID NO: 1, or a functional variant thereof, is to an alanine or valine residue.

23. The fusion protein of claim 22, wherein the substitution at residue 43 and/or 67 is to an alanine residue and the substitution at residue 70 is to a valine.

24. The fusion protein of claim 6, wherein the IL-38 polypeptide comprises:
i) a serine residue at, or at a position equivalent to residue 37 of SEQ ID NO: 1 or a functional variant thereof;
an arginine residue at, or at a position equivalent to residue 38 of SEQ ID NO: 1 or a functional variant thereof;
iii) an alanine residue at, or at a position equivalent to residue 43 of SEQ ID NO: 1 or a functional variant thereof;
iv) an alanine residue at, or at a position equivalent to residue 67 of SEQ ID NO: 1 or a functional variant thereof;
v) a valine residue at, or at a position equivalent to residue 70 of SEQ ID NO: 1 or a functional variant thereof; and/or
vi) a serine residue at, or at a position equivalent to residue 123 of SEQ ID NO: 1.

25. The fusion protein of claim 6, wherein the IL-38 polypeptide comprises the amino acid substitutions C70V, C37S, C38R, C43A, C67A, and C123S (numbered according to SEQ ID NO: 1).

26. The fusion protein of any one of claims 1 to 25, wherein the Fc region of the antibody of the fusion protein is an Fc region of IgG1.

27. The fusion protein of any one of claims 1 to 26, wherein the IL-38 polypeptide, comprises an amino acid sequence having a mutation or modification that reduces the capacity of the polypeptide to form a dimer compared to an IL-38 polypeptide having the amino acid sequence of SEQ ID NO: 1.

28. The fusion protein of claim 27, wherein the mutation is not A51D.

29. The fusion protein of claim 27, wherein the mutation prevents the IL-38 polypeptide from forming a dimerization interface that enables dimerization of IL-38 monomers.

30. The fusion protein of claim 29, wherein the mutation is located in a region of the IL-38 polypeptide that has the same amino acid sequence as the amino acid sequence that forms the dimerization interface of an IL-38 monomer.

31. The fusion protein of claim 30, wherein the mutation is located in the loop between the 34 and 35 strands that form the dimerization interface of an IL-38 monomer.

32. The fusion protein of any one of claims 1 to 31, wherein the amino acid sequence of the IL-38 polypeptide has at least 80%, 81%, 82%, 83%, 84%, 85%, 86%, 87%, 88%, 89%, 90%, 91%, 92%, 93%, 94%, 95%, 96%, 97%, 98% or 99% sequence identity with the amino acid sequence set forth in SEQ ID NO: 1 and has mutation or modification at any one or more residues, or residues equivalent to 43 to 58 of SEQ ID NO: 1.

33. The fusion protein of claim 32, wherein the mutation or modification is located within a region, or a region equivalent to, residues G49, L50, A51, T53 or V55 of SEQ ID NO: 1.

34. The fusion protein of claim 32, wherein the IL-38 polypeptide comprises an amino acid sequence wherein the amino acid residue at, or equivalent to:
position 43 in SEQ ID NO: 1 is not a Cysteine;
position 44 in SEQ IS NO: 1 is not an Isoleucine;
position 45 in SEQ ID NO: 1 is not a Leucine;
position 46 in SEQ ID NO: 1 is not a Proline;
position 47 in SEQ ID NO: 1 is not an Asparagine;
position 48 in SEQ ID NO: 1 is not an Arginine;
position 49 in SEQ ID NO: 1 is not a Glycine, preferably not a proline;
position 50 in SEQ ID NO: 1 is not a Leucine, preferably not an aspartate or glutamine;
position 51 in SEQ ID NO: 1 is not an Alanine;
position 52 in SEQ ID NO: 1 is not an Arginine;
position 53 in SEQ ID NO: 1 is not a Threonine, preferably not deleted;
position 54 in SEQ ID NO: 1 is not a Lysine;
position 55 in SEQ ID NO:1 is not a Valine;
position 56 in SEQ ID NO: 1 is not a Proline;
position 57 in SEQ ID NO: 1 is not an Isoleucine; and
position 58 in SEQ ID NO: 1 is not a Phenylalanine.

35. The fusion protein of any one of claims 32 to 34, wherein the mutation is a replacement with a non-conservative amino acid.

36. The fusion protein of any one of claims 32 to 35, wherein the mutation is replacement with alanine or an amino acid with an opposite charge.

37. The fusion protein of any one of claims 32 to 36 wherein the mutation is any one of T53N, V55Q, or V55T.

38. The fusion protein of any one of claims 32 to 37, wherein the IL-38 polypeptide exists in a slower equilibrium between monomer and dimer than an IL-38 polypeptide having the sequence of SEQ ID NO: 1.

39. The fusion protein of any one of claims 1 to 38, wherein the IL-38 polypeptide comprises an amino acid sequence as set forth in any one of SEQ ID NOs: 1 or 2.

40. The fusion protein of any one of preceding claims wherein the IL-38 polypeptide comprises an N terminal truncation.

41. The fusion protein of 40, wherein the N terminal truncation is of, or equivalent to, residues 1, residues 1 to 2, 1 to 3, 1 to 4, 1 to 5, 1 to 6, 1 to 7, 1 to 8 or more of SEQ ID NO: 1 up to residues 1 to 20.

42. The fusion protein of claim 41, wherein the N-terminal truncation is of, or equivalent to, residues 1 to 2 or SEQ ID NO: 1.

43. The fusion protein of claim 42, wherein the IL-38 polypeptide comprises an amino acid sequence as set forth in any one of SEQ ID NOs: 4 to 8 or 13 to 17 or 24.

44. The fusion protein of claim 41, wherein the IL-38 polypeptide comprises the amino acid sequence as set forth in SEQ ID NO: 10 or 11.

45. The fusion protein of claim 41, wherein the IL-38 polypeptide comprises the amino acid sequence as set forth in SEQ ID NO: 12.

46. The fusion protein of any one of the preceding claims, wherein the IL-38 polypeptide and the Fc region of an antibody are joined via a linker region.

47. The fusion protein of any one of the preceding claims, wherein the fusion protein comprises the amino acid sequence as set forth in any one of SEQ ID NOs: 21, 23, 25 or 27.

48. The fusion protein of any one of claims 1 to 47, wherein the fusion protein inhibits the production or function of an inflammatory cytokine, chemokine or transcription factor involved in inflammation.

49. The fusion protein of any one of claims 1 to 48, wherein the fusion protein reduces the production of IRF3/7, RANTES, IL-6, IFNL, IFNB, ISG15 and/or CXCL10.

50. A pharmaceutical composition for treating or preventing an inflammatory disease or condition comprising a fusion protein of any one of claims 1 to 49 and a pharmaceutically acceptable diluent, excipient or carrier.

51. A pharmaceutical composition according to claim 50, wherein 75%, 80%, 81%, 82%, 83%, 84%, 85%, 86%, 87%, 88%, 89%, 90%, 91%, 92%, 93%, 94%, 95%, 96%, 97%, 98% or 99% of the IL-38 present in the composition is monomeric.

52. A method of inhibiting inflammation in a subject in need thereof, the method comprising administering to the subject a therapeutically effective amount of a fusion protein or pharmaceutical composition of any one of claims 1 to 51, thereby inhibiting inflammation in the subject.

53. A method of treating or preventing an inflammatory disease or condition in a subject in need thereof, the method comprising administering to the subject a therapeutically effective amount of a fusion protein or pharmaceutical composition of any one of claims 1 to 51, thereby treating or preventing an inflammatory disease or condition in a subject.

54. A method of alleviating or ameliorating a symptom of an inflammatory disease or condition in a subject in need thereof, the method comprising administering to the subject in need thereof a therapeutically effective amount of a fusion protein or pharmaceutical composition of any one of claims 1 to 51, thereby alleviating or ameliorating a symptom of an inflammatory disease or condition in the subject.

55. Use of a therapeutically effective amount of a fusion protein of any one of claims 1 to 49, in the manufacture of a medicament for the treatment or prevention of an inflammatory disease or condition in a subject in need thereof.

56. The method or use of any one of claims 52 to 55, wherein the inflammatory disease is an autoimmune disease.

57. The method or use of any one of claims 52 to 55, wherein the inflammatory disease is systemic lupus erythematosus (SLE).

58. The method or use of any one of claims 52 to 55 wherein the subject is homozygous wild-type (genotype C; C) or heterozygous (genotype C; A) at SNP locus rs6743376.

59. A nucleic acid molecule encoding a fusion protein according to any one of claims 1 to 49.

60. A vector comprising a nucleic acid molecule according to claim 59.

61. A cell comprising a vector according to claim 60 or nucleic acid molecule according to claim 59.


SEQUENCE LISTING (27 sequences)

SEQ ID NO: 1 - protein, 152 aa, synthetic construct
MCSLPMARYYIIKYADQKALYTRDGQLLVGDPVADNCCAEKICILPNRGLARTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 2 - protein, 152 aa, synthetic construct [sequence omitted]

SEQ ID NO: 3 - protein, 31 aa, synthetic construct
MSSSFLPEPLPAKSLQHGVPLSLDSSLSSLL

SEQ ID NO: 4 - protein, 150 aa, synthetic construct
SLPMARYYIIKYADQKALYTRDGQLLVGDPVADNCCAEKICILPNRGLARTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 5 - protein, 150 aa, synthetic construct
SLPMARYYIIKYADQKALYTRDGQLLVGDPVADNCCAEKICILPNRGLDRTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 6 - protein, 150 aa, synthetic construct
SLPMARYYIIKYADQKALYTRDGQLLVGDPVADNCCAEKICILPNRGLARNKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 7 - protein, 150 aa, synthetic construct
SLPMARYYIIKYADQKALYTRDGQLLVGDPVADNCCAEKICILPNRGLARTKTPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 8 - protein, 150 aa, synthetic construct
SLPMARYYIIKYADQKALYTRDGQLLVGDPVADNCCAEKICILPNRGLARTKQPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 9 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 147 aa, synthetic construct [sequence omitted]

SEQ ID NO: 11 - protein, 147 aa, synthetic construct
MARYYIIKYADQKALYTRDGQLLVGDPVADNCCAEKICILPNRGLDRTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 12 - protein, 133 aa, synthetic construct
LYTRDGQLLVGDPVADNCCAEKICILPNRGLDRTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 13 - protein, 150 aa, synthetic construct
SLPMARYYIIKYADQKALYTRDGQLLVGDPVADNCSAEKICILPNRGLDRTKVPIFLGIQGGSRCLACVETEEGPSLQLEDVNIEELYKGGEEATRFTFFQSSSGSAFRLEAAAWPGWFLSGPAEPQQPVQLTKESEPSARTKFYFEQSW

SEQ ID NO: 14 - protein, 150 aa, synthetic construct
SLPMARYYIIKYADQKALYTRDGQLLVGDPVADNSSAEKICILPNRGLDRTKVPIFLGIQGGSRCLACVETEEGPSLQLEDVNIEELYKGGEEATRFTFFQSSSGSAFRLEAAAWPGWFLSGPAEPQQPVQLTKESEPSARTKFYFEQSW

SEQ ID NO: 15 - protein, 150 aa, synthetic construct; identical to SEQ ID NO: 14

SEQ ID NO: 16 - protein, 150 aa, synthetic construct
SLPMARYYIIKYADQKALYTRDGQLLVGDPVADNSSAEKISILPNRGLDRTKVPIFLGIQGGSRCLACVETEEGPSLQLEDVNIEELYKGGEEATRFTFFQSSSGSAFRLEAAAWPGWFLSGPAEPQQPVQLTKESEPSARTKFYFEQSW

SEQ ID NO: 17 - protein, 150 aa, synthetic construct
SLPMARYYIIKYADQKALYTRDGQLLVGDPVADNSSAEKIAILPNRGLDRTKVPIFLGIQGGSRCLACVETEEGPSLQLEDVNIEELYKGGEEATRFTFFQSSSGSAFRLEAAAWPGWFLSGPAEPQQPVQLTKESEPSARTKFYFEQSW

SEQ ID NO: 18 - protein, 383 aa, synthetic construct
SLPMARYYIIKYADQKALYTRDGQLLVGDPVADNSRAEKIAILPNRGLARTKVPIFLGIQGGSRALAVVETEEGPSLQLEDVNIEELYKGGEEATRFTFFQSSSGSAFRLEAAAWPGWFLSGPAEPQQPVQLTKESEPSARTKFYFEQSWGSGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 383 aa, synthetic construct
SLPMARYYIIKYADQKALYTRDGQLLVGDPVADNSRAEKIAILPNRGLDRTKVPIFLGIQGGSRALAVVETEEGPSLQLEDVNIEELYKGGEEATRFTFFQSSSGSAFRLEAAAWPGWFLSGPAEPQQPVQLTKESEPSARTKFYFEQSWGSGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 21 - protein, 402 aa, synthetic construct
MGWSCIILFLVATATGVHSSLPMARYYIIKYADQKALYTRDGQLLVGDPVADNSRAEKIAILPNRGLARTKVPIFLGIQGGSRALAVVETEEGPSLQLEDVNIEELYKGGEEATRFTFFQSSSGSAFRLEAAAWPGWFLSGPAEPQQPVQLTKESEPSARTKFYFEQSWGSGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 246 aa, synthetic construct
MGWSCIILFLVATATGVHSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 23 - protein, 396 aa, synthetic construct
MGWSCIILFLVATATGVHSSLPMARYYIIKYADQKALYTRDGQLLVGDPVADNSRAEKIAILPNRGLARTKVPIFLGIQGGSRALAVVETEEGPSLQLEDVNIEELYKGGEEATRFTFFQSSSGSAFRLEAAAWPGWFLSGPAEPQQPVQLTKESEPSARTKFYFEQSWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 150 aa, synthetic construct
SLPMARYYIIKYADQKALYTRDGQLLVGDPVADNSRAEKIAILPNRGLARTKVPIFLGIQGGSRALAVVETEEGPSLQLEDVNIEELYKGGEEATRFTFFQSSSGSAFRLEAAAWPGWFLSGPAEPQQPVQLTKESEPSARTKFYFEQSW

SEQ ID NO: 25 - protein, 383 aa, synthetic construct; identical to SEQ ID NO: 18

SEQ ID NO: 26 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 27 - protein, 377 aa, synthetic construct
SLPMARYYIIKYADQKALYTRDGQLLVGDPVADNSRAEKIAILPNRGLARTKVPIFLGIQGGSRALAVVETEEGPSLQLEDVNIEELYKGGEEATRFTFFQSSSGSAFRLEAAAWPGWFLSGPAEPQQPVQLTKESEPSARTKFYFEQSWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK